SEQUENCE LISTING (17 sequences)

SEQ ID NO: 1 - DNA, 58 bp, Artificial Sequence
  Synthetic Primer
agtacgtattgacaattaatcatccggctcgtataatattaaagaggagaaacatatg

SEQ ID NO: 2 - DNA, 47 bp, Artificial Sequence
  Synthetic Primer
taatggatccaagatctcgaattctaagcttcgatatcgactagtag

SEQ ID NO: 3 - DNA, 28 bp, Artificial Sequence
  Synthetic Primer
agcgatgcgaatattcatgccgactaac

SEQ ID NO: 4 - DNA, 43 bp, Artificial Sequence
  Synthetic Primer
gatgattaattgtcaaatcgatgccgaaatcatggctacaatc

SEQ ID NO: 5 - DNA, 43 bp, Artificial Sequence
  Synthetic Primer
gattgtagccatgatttcggcatcgatttgacaattaatcatc

SEQ ID NO: 6 - DNA, 49 bp, Artificial Sequence
  Synthetic Primer
agattttgagacacaacgtggctttactagtcgatatcgaagcttagaa

SEQ ID NO: 7 - DNA, 49 bp, Artificial Sequence
  Synthetic Primer
ttctaagcttcgatatcgactagtaaagccacgttgtgtctcaaaatct

SEQ ID NO: 8 - DNA, 44 bp, Artificial Sequence
  Synthetic Primer
aatttgatcgatgtagggactgctgattagaaaaactcatcgag

SEQ ID NO: 9 - DNA, 44 bp, Artificial Sequence
  Synthetic Primer
ctcgatgagtttttctaatcagcagtccctacatcgatcaaatt

SEQ ID NO: 10 - DNA, 33 bp, Artificial Sequence
  Synthetic Primer
ggagttggatattttccatttgttgagcgacct

SEQ ID NO: 11 - DNA, 29 bp, Artificial Sequence
  Synthetic Primer
gtcttcgtcaggaaatgttgatggcgatc

SEQ ID NO: 12 - DNA, 25 bp, Artificial Sequence
  Synthetic Primer
gggcttcgaggttcggcacaattaa

SEQ ID NO: 13 - DNA, 1809 bp, Aquifex pyrophilus
atatcgatttgacaattaatcatccggctcgtataatattaaagaggagaaacatatggtgattacccgaaatttgaaacacatctccgggaacaccgaaaggatcattaaattatcctctgaggaatatctcttgattggtgaatatactcttcaggacatcaaaacctttaagaccctcgaaaacgtaatttatatcgcgttgcccatgaaatacagtcccctgctcgataaagtggcgctttacacccggctaacggacttaatgcaggatacgaacctgagcgaatataaagggcggggtgtgatcatcggtattgcggattccggtattgattacacccacccggcattccgtggccgtattctggaaattcacgacctgaccactggcgaggtttgctacaaagaagaaattgagaaagggaactgcaaccaacgcgacaccgacggccacggtacccatgtggcagggatcgcggctggtgaccatgaggtctatcgtggaattgctcccgaagcggaactgattgtcgtgaaaatcggcaatgaagaatttcgtgaagacaaaattattgaagctctcgacatcctagccaaacgtattaaagcttatggtaaacccggcgttatcaacctcagcctaggcactcccctgggtccccatgacggcaccgatccaatttcccggttcattgagtccattgtaaacacctataaaattcccgtcgttgtggctgccggcaactggggcaatgagcccattcacgataaattgaacttgttccaggagaatacctccagcttcgatgttattggctcctgggcctacgtggacatttggattatgggaaccgatagtgtggaactgaccatcaattccccttgtcaagcggtgtccattcgggaaggcgataccaaattgatcgacctaggcaactgtggcactctggccgttagttttagcggtcgcaaccctcttaacggtgataaaaacgtattcattaaaatcgacaacgtgaatgagaactttcggaacgggtggagtgtctcctttactcccattgatgtgaaagacggtgaagtgcacctgtggggtgaaaatgtagcttttgcgagccccgactacagctataccatcagcagcttagctagctccagctccgtcatcagcgtggggtctttcaccagcaaggtcaacgtcaacattaatccctactcgatcttggggagcatttcgaatttctcctcgattggccctagccggcggtgttcctacggttgtaaagagaacttgaaacccgacatcgtagcacccggggagttggtgtgctccgcgtaccctctcaacattaagccgttgattgacttatgcttttacgaaggtttcaattggatgcaagggacctctatgtctgcccccgcagtgaccggtgctgttgccttgttgttgcaagttgagcataccttgaccccctccgaagtgaaagaaatccttatcaataatgcttataaagactcctttaccactcaaaaacctaacaatgtttacggtcacggcaagctggacatttacaagtctgttagcagtttgttagaagaacgggaaggtttttccgaccgcaataccacggaacgtgaaaccctcaatgtttcatctggtggcggtgggtgcaccagccaagggcaagtgtcaatcatctacttgatcaccttcgccctctttttgaaagtgattcgttttttataccgctttcttttttaatggatccaagatctcgaattctaagcttcgatatcgactagtag

SEQ ID NO: 14 - DNA, 1785 bp, Unknown
  Thermoanaerobacter tengcongensis
atatcgatttgacaattaatcatccggctcgtataatattaaagaggagaaacatatgaaaaaacaccaattggctaagattctgctgtcccttgcgttgatcattagcctcatttccttaaatgagattttggttcaagcccaacccaaccaaatcaacctgccgatagacagccccagtaaaatctacccatccctgccccaaaaaatctccatgcaagactccaacaaaaataaaatttttgatgatttggagcaacgacttctgaataaacccgattccgaggaatttccagtaattattacctttaacaagcccgtatccgatgctgatatctttactattgctaagaacattggcaaattcaatatcaagcatcggtacaaaattatccccagcattgctgccaatctcaccaagagtcaaatcaacgttctatccaaattggaaatcgtcaaacaaatcgagtacgatgaacccgtatatgcaaccttggataccgccacgaagtggttcggtattactaaggcccggtctgacttcggagttaccggcaagaacattaccattgccatcattgacacaggtattgatggcaaccacgttgatctcagcggaggtaagattatcgggtggaaagacttcattaacaataaaaccaccccttatgatgacaatggtcacggtactcacgtcgctagtatcgccgctggtaccggcgctggtaactctttttataaaggcgtggcccctgatgccttgctcgttgggattaaagtcctcgatgcaaatggttccggcagcatgtccactgttaccgcgggtattgattgggccgtgcaaaataaagatgtttatggcatcaaagtgattaacctgtccttggggacctccacgtccagcgacgggaccgatagtacctccctagccgttaaccgtgctgtggacagcggcattgtagtggttgttgctgcgggtaacagtgggcccgcaaaatacactattggttcccccggtgcggcggaaaaagcgatcaccgtggccgctatggccgacgttggcgagctgggtttcaacttagccagcttcagttcccgtggacccaccgccgatggccggatcaaacccgatatcgccgcccccggctataacattaccgctgccaaagctaattccgtgaatggctatgttacctactctgggacgtccatggccactcccttcgttgcgggtaccgtcgccttgatgctcaacgccaaccctaatctgacccccaacgatgccaagaacatcatcatgtccacggccaaaagttggggccctccctctaaaaacgtggattatggtgccggtcgtctggacggatacgaagccattcgtgtggccggcaacttccgtggtaataacattgatgtgcccaaccactactacatttccggttacttacccgggagccggtattccgacacctggacttttaatgccaccaatacttcttaccccattgccattaccctcattatccccgactgggccaattataatccggattttgatatttacttgtatgatcctagtggaaccttaattaaatcctccaccggcacgcaacggcaggaaaccatcactattttaccctcccaaaccggcacttattatgttaaggtttattcttaccgtgggtccggcaactattttttcgacctctccgcgggtggtagcgaaccagttaagtactaatggatccaagatctcgaattctaagcttcgatatcgactagtag

SEQ ID NO: 15 - DNA, 1188 bp, Unknown
  Thermoactinomyces
atatcgatttgacaattaatcatccggctcgtataatattaaagaggagaaacatatgtttgccgcttctcccgcgagcaccgacgactatgtacccggcgaacttatcgtgaagttcaaagatggcattagtgctcaatccacccaatctattcatgcgcaatatggtgctaaatccattgaaaaaagtaaatacttgggctttgaagtcgtgaaattcgatggttctgtcgaaaaaatgattgaaaaatataagaataaccctaacgtagaatatgtggaacccaaccattatgtacatattatgtggacccccaacgatttaaccagccgtcaatggggtccccaaaaagtgcaggcgccacaggcatgggacgtaactcgaagctcctcctctaccgtcattgcgattgtagataccggtgtccagaccaatcatcccgacctacaaggcaaaattgtgcagggatatgattttgtggataatgatagcaacccacaagacggaaatggtcatggcactcactgtgcaggaattgcggctgccgttaccaacaatgggaccgggatcgccggcatggcccccaatgcctccatcatgcccgtacgcgttttgaacaacagcggtagtggtactatggccgccgtcgccaatggaattgcctacgcggcccaaaacggtgccgacgttatttccttgtccctaggtggtacctccggttccagcgccttgcaaagcgctgtgcaacaagcctggaactctggcgccgtagtggtcgccgccgctgggaattcctccagtagtacccccaattatcccgcgtattattctcaagctatcgcagtagcgtccaccgatagcaatgattctttatcctatttctccaattacggttcctgggtggatgttgccgcccccggttctaatatctattccacgtatctcaactcctcctatgctagtctgtccgggacttcaatggccacaccccacgtggctggtctcgcggctctcctagcttctcaaggacggagcaacagtcaaattcgtgccgccatcgaaaacacggctgacaaaatctctggcaccggcacctacttccaacacggtcgtatcaacgcttataaggccgtgaactattaatggatccaagatctcgaattctaagcttcgatatcgactagtag

SEQ ID NO: 16 - DNA, 1305 bp, Unknown
  Geobacillus stearothermophilus
atatcgatttgacaattaatcatccggctcgtataatattaaagaggagaaacatatgaaatttaaagccattgtgtccctgagcctggctgtgtccatgtccttgttcccgtttctggtagaagccgccagcaacgacggcgtggaatccccaaaaaccgtaagtgaaattaatgtcagtcacgaaaaaggtgcttatgtccagggcgaagttatcgttcagttcaaagaacaggttaacgctgaagaaaaggcaaaagcgcttaaagaagttggtgctacggccgtgcccgacaatgaccgcgtgaaatccaaattcaacgtgttaaaggtaggaaacgtggaagctgtggttaaagcgctgaaccacaaccccctcgtggagtacgccgaaccgaattatttgttcaatgccgcgtggacgcctaatgatacctattatcagggctaccaatacggaccccagaatacctacaccgcttacgcctgggacgtcactaagggctctagcggtcaggaaatcgcagtgattgataccggcgttgactacacccaccctgacctggacggcaaagtaattaagggatacgattttgtcgacaacgattatgatcccatggatctcaacaaccacgggacccacgttgccggcattgcggctgcggagactaataacgcgaccggaatcgccggtatggcacccaatacgcggatcttagcggtgcgggctcttgatcgcaacggttccggcaccttgtccgatattgccgacgcgatcatttatgcggccgattctggcgctgaagtgatcaacctcagtctggggtgcgattgccacaccactacgttggaaaatgctgttaactatgcttggaataaaggttccgtggtggtggctgccgccggcaataacggttccagcaccacctttgaacccgcctcctacgagaacgtaattgcagtgggtgctgtggatcaatacgaccgcctcgcctccttttcaaattacggcacctgggtcgatgtggtcgctcctggtgtggatattgtgagtaccattaccggtaaccggtatgcctatatgagcgggacctccatggcgtctcctcacgtggccggtctggctgctctgctggcctcccagggccgtaataacatcgaaattcgacaagccatcgaacaaactgctgataaaattagtggcaccggtacctatttcaaatatgggcgtatcaattcctataacgccgtcacctactaatggatccaagatctcgaattctaagcttcgatatcgactagtag

SEQ ID NO: 17 - DNA, 2880 bp, Artificial Sequence
  Synthetic Primer
tctgatatcgtttttatttgttaactgttaattgtccttgttcaaggatgctgtctttgacaacagatgttttcttgcctttgatgttcagcaggaagcttggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacctccaggccttttgcagaagagatatttttaattgtggacgaatcgaattcaggaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcgatcctctagctagagtcgacctgcatttaaataaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcaatttaaatgagatatctgca

(19) United States
(12) Patent Application Publication
(10) Pub. No.: US 2018/0002656 A1
(43) Pub. Date: Jan. 4, 2018

(54) METHODS AND COMPOSITIONS FOR HIGH-EFFICIENCY PRODUCTION OF BIOFUEL AND/OR BIOMASS

(71) Applicant: SABIC Global Technologies B.V., Bergen op Zoom (NL)

(72) Inventors: Xinyao Liu, Thuwal (SA); Duncan Rowe, Thuwal (SA)

(21) Appl. No.: 15/545,372

(22) PCT Filed: Nov. 30, 2015
(86) PCT No.: PCT/IB2015/059227

§ 371 Date: Jul. 21, 2017

Related U.S. Application Data
(60) Provisional application No. 62/108,917, filed on Jan. 28, 2015.
(60) Provisional application No. 62/258,785, filed on Nov. 23, 2015.

(51) Int. Cl. C12N 1/06; C12N 9/52; C12N 1/20

(52) U.S. Cl. CPC C12N 1/06; C12N 1/20; C12N 9/52

(57) ABSTRACT

Methods and composition related to genetically modified cells for producing intracellular biological products are provided. A method can include genetically engineering of cells to express a thermostable protease. As one advantage, the cells may be suitable for producing biological products with improved efficiency.

### CROSS-REFERENCE TO RELATED APPLICATIONS

This application claims the benefit of priority of U.S. Provisional Patent Application No. 62/108,917 filed Jan. 28, 2015, and U.S. Provisional Patent Application No. 62/258,785 filed Nov. 23, 2015. The entire contents of each of the above-referenced disclosures are specifically incorporated herein by reference without disclaimer.

### BACKGROUND OF THE INVENTION

### A. Field of the Invention

The present invention relates generally to the field of genetic engineering. More particularly, it concerns making and using engineered cells for obtaining biofuel and/or biomass, particularly intracellular biological products.

### B. Description of Related Art

Cell disruption is often required for recovering intracellular products from cells such as bacteria to release intracellular products for further separation processes. Cell disruption methods that have been used include bead milling, high-pressure homogenizers, autoclaving, sonication, super critical CO₂ extraction, enzymatic cell wall degradation and addition of hydrochloric acid, sodium hydroxide, or alkaline lysis. All these processes are cost intensive at large-scale (e.g., about $1.75 for extracting 1 gallon algal oil). Taking enzymatic digestion by adding external enzymes to cells as an example, high cost of enzymes stems from their production and from the fact that the enzymes usually cannot be recovered and recycled after they are used.

### SUMMARY OF THE INVENTION

The present disclosure concerns methods and compositions relating to microbial organisms and their use in the manufacture of valuable resources. In particular, the microbial organisms are valuable in the manufacture of biofuel, biomass, and intracellular biological products. While not limited to use as biomass, the ability to use microbial cells as biomass is beneficial, particularly where the microbes can be controlled under conditions that minimize the cost to yield intracellular products from the microbial cells. Compositions include cells that have a non-native nucleic acid encoding a protease that is more active under certain conditions than others. In particular embodiments, the protease remains active under high temperature conditions, e.g., at a temperature above 50° C.

In some embodiments, there are compositions that include one or more recombinant and/or engineered microbial cells can include a heterologous nucleic acid segment encoding a thermostable protease. In certain embodiments, a thermostable protease has protease activity at a temperature between 46° C. and 60° C., or between 48° C. and 55° C.

In particular embodiments, the a heterologous nucleic acid segment is integrated into the genome or into a chromosome of the microbial organism, which means it is connected with the genome of the organism through covalent bonds. A typical covalent bond would be through phosphodiester bonds between nucleotides. In certain embodiments, the nucleic acid segment will be integrated into a cell's nuclear genome, though it is possible for it to be integrated into the mitochondrial genome. In other embodiments, the heterologous nucleic acid segment is not integrated into the genome, in which case it is in the cell extrachromosomally, such as on a plasmid. In certain instances, the heterologous nucleic acid segment is integrated into the genome of the organism at a random location, while in other embodiments, it is integrated into the genome at a planned or specified location. In some embodiments, the heterologous nucleic acid segment is integrated into the genome at a location encoding a protease or in a location that does not encode an essential or housekeeping gene or other coding sequence. In some embodiments, any one of the integration methods and/or locations may be excluded as to the claimed invention.

In certain embodiments, the thermostable protease is one of six broad groups of proteases based on the catalytic residue it uses: a serine-type protease (uses a serine alcohol; threonine protease (uses a threonine secondary alcohol); a cysteine protease (uses a cysteine thiol); an aspartate protease (uses an aspartate carboxylic acid); a glutamic acid protease (uses a glutamate carboxylic acid), and, a metalloprotease (uses a metal, usually zinc). In specific embodiments, the thermostable protease is not a bacteriophage thermostable protease. In particular embodiments, the thermostable protease is from a bacteria. In other embodiments the thermostable protease is from a fungus, such as from yeast. In certain embodiments, the thermostable protease is from a thermophilic organism, which refers to an organism that thrives at relatively high temperatures, such as between 41° C. and 122° C. In certain embodiments, thermostable protease is from a thermophilic bacteria such as a eubacteria. In some embodiments, any one of the proteases disclosed herein may be excluded as to the claimed invention.

In particular embodiments, the thermostable protease is from Aquifex pyrophilus, Thermoanaerobacter tengcongensis, Thermoactinomyces sp. E79, Thermomonospora fusca, Geobacillus stearothermaphilus F1, Pyrococcus furiosus DSM3638, Bacillus caldolytics, Sulfolobus acidocaldarius, Bacillus stearothermaphilus, Thermus thermophilus HB8, Alicyclobacillus sendaiensis, Chaetomium thermophilum, Bacillus stearothermophilus, Aspergillus fumigatus WY-2, Bacillus subtilis WB30, or Thermoascus aurantiacus. In specific embodiments, the thermostable protease is App (Aquifex pyrophilus), Ttp (Thermoanaerobacter tengcongensis), Tap (Thermoactinomyces sp. E79), Tfp (Thermomonospora fusca), gsp (Geobacillus stearothermaphilus F1), PFUL (Pyrococcus furiosus DSM3638), npr (Bacillus caldolytics), stp (Sulfolobus acidocaldarius), nprM (Bacillus stearothermaphilus), TtHB (Thermus thermophilus HB8), scpA (Alicyclobacillus sendaiensis), pro (Chaetomium thermophilum), bstp (Bacillus stearothermophilus), Afp (Aspergillus fumigatus WY-2), nprB (Bacillus subtilis WB30), or tau (Thermoascus aurantiacus). In some embodiments, any one of these proteases may be excluded as to the claimed invention.

In particular embodiments, there is also a selectable or screenable marker. The selectable marker may be one that can be used for negatively selecting for a cell with a particular characteristic or one that may be used for positively selecting for a cell with a particular characteristic. In certain embodiments, there may be more than one selectable or screenable marker on the nucleic acid segment or in the cell. In particular embodiments, the selectable or screenable marker may be encoded by the same nucleic acid segment as a heterologous protease. In some embodiments, any one of the selectable and/or screenable marker disclosed herein may be excluded as to the claimed invention.

In some embodiments, the thermostable protease is under the transcriptional control of a promoter, which may be the native promoter of the thermostable protease, or the promoter may be heterologous with respect to the thermostable protease. Even if the promoter is from the thermostable protease, it may be only part of the native promoter in some embodiments. In other embodiments, the promoter may be a promoter that is native to the cell but for another gene. In some cases, the promoter is constitutive or it may be from a housekeeping gene. In other embodiments, the promoter may be inducible or it may be controllable by external stimuli. In some embodiments, any one of the promoters disclosed herein may be excluded as to the claimed invention.

The cell containing the heterologous thermostable protease is a microbial cell. In some embodiments, the cell is prokaryotic, while in others it is eukaryotic. In some cases, the cells are bacteria, yeast, or algae. In certain embodiments, it is a bacteria cell that is photosynthetic, meaning it uses the sun as a source of energy. A photosynthetic cell uses the process of photosynthesis. In certain embodiments, the cell is one that uses light as its sole energy source, uses light as an energy source in the presence of air, and/or produces oxygen. In particular embodiments, the cell is a cyanobacteria. In additional embodiments, cells that grow in the light but primarily only anaerobically are excluded. In further embodiments, the cells are algae or microalgae. In some embodiments, any one of the cells disclosed herein may be excluded as to the claimed invention.

In one embodiment, there is an engineered cyanobacteria cell that includes an integrated, heterologous nucleic acid segment encoding at least one selectable marker and at least one thermostable protease under the control of a constitutive promoter. In another embodiment, there is an inducible, self-destructing engineered photosynthetic cell that includes a heterologous nucleic acid segment encoding at least one selectable marker and at least one thermostable protease.

In another embodiment there is an engineered or recombinant photosynthetic bacteria cell that includes a heterologous nucleic acid segment encoding a thermostable protease. It is understood that progeny of such cells are also “engineered” or “recombinant” so long as they have the heterologous nucleic acid segment (which may be integrated into the genome or maintained extrachromosomally).

It is contemplated that compositions may contain a plurality of such cells containing a heterologous nucleic acid segment encoding a thermostable protease. In some instances, a composition may include bacteria media or water; in some embodiments, it may include a container for growing the cells.

In various embodiments, the number of bacteria is at least or at most or between about 10⁸, 10⁹, 10¹⁰, 10¹¹, 10¹², 10¹³, 10¹⁴, 10¹⁵, 10¹⁶, 10¹⁷, 10¹⁸, 10¹⁹, 10²⁰, 10²¹, 10²², 10²³, 10²⁴, 10²⁵, 10²⁶, 10²⁷, 10²⁸, 10²⁹, 10³⁰, 10³¹, 10³², 10³², 10³², 10³³, 10³⁴ cells (or any range derivable therein) or at least or at most 1, 2, 3, 4, 5, 6, 7, 8, 9, 10, 11, 12, 13, 14, 15, 16, 17, 18, 19, 20, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 tons (or any range derivable therein) of biomass.

In some cases, a composition does not include more than a contaminating amount of nickel. In certain embodiments, nickel has not been purposely added to the composition; in some embodiments, there is less than 0.1 grams of nickel per liter of liquid that can be measured in the composition.

In certain embodiments, there is a cell lysate that may or may not contain any cells that are unlysed. In some embodiments, a cell lysate comprises lysed cells that are described herein. In further embodiments, a composition comprises lysed photosynthetic bacteria cells that contained a nucleic acid segment encoding a heterologous thermostable protease. In some embodiments, at least 30, 40, 50, 60, 70, 80, 90% or more of the cells (or any range derivable therein) are lysed. Alternatively, a container or mixture of cells may be characterized as containing fewer than or at most 10, 20, 30, 40, or 50% intact cells (or any range derivable therein).

Other embodiments include method of using such cells and cell lysates. In some embodiments, there are methods for inducing lysis of photosynthetic bacteria cells, methods for generating inducible, self-destructing engineered photosynthetic bacteria, methods for creating an inducible, self-destructing engineered photosynthetic bacteria, methods for providing nutrients from cell lysate, methods for generating lysate from cells, methods for extracting cellular compositions or components, methods for improved extractability of cellular compositions, methods for purifying biomass from cells, and methods for isolating cellular components from engineered cells. In some embodiments, any one of the methods of using such cells and cell lysates disclosed herein may be excluded as to the claimed invention.

Methods and products by process may include exposing cells or heating cells to a certain temperature and/or for a certain amount of time. In some embodiments, there are steps of exposing cells to an elevated temperature between about 45° C. and about 100° C.; exposing cells to an elevated temperature for at least 1 hour; exposing cells to an elevated temperature between 2 hours and 48 hours; exposing cells to an elevated temperature between about 45° C. and about 80° C.; and/or, exposing cells to an elevated temperature of not more than about 60° C. In particular embodiments, the cells are exposed to the sun or are heated by solar heat. In other embodiments, the cells are heated by a mechanical device. In some embodiments, any one of the temperatures and/or amounts of time disclosed herein may be excluded as to the claimed invention.

In some embodiments, the cells are enclosed or held in a fabricated container. The container may be made of metal, plastic, or glass, or a combination thereof. In certain embodiments, the container is capable of holding up to or not more than 5, 10, 15, 20, 25, 30, 35, 40, 45, 50, 55, 60, 65, 70, 75, 80, 85, 90, 95, 100, 200, 300, 400, 500, 600, 700, 800, 900, 1000 or more gallons or kiloliters (or any range derivable therein).

Methods may also involve creating and/or collecting cell lysate from the cells. In some embodiments, cell lysate is creating by exposing the cells to an elevated temperature and not by exposure to exogenous chemicals or by asserting physical force.

Another step that may be included in methods described herein is culturing the cells in a compatible media to promote growth or in water. The media or water may be maintained or be at a particular pH such as about, at least about, or at most about 5, 5.5, 6, 6.5, 7, 7.5, 8, 8.5, or 9. In some instances, the water is a body of water, such as a lake or river. Cells may be cultured with or without a selective agent. In certain embodiments, the cells are cultured with a selective agent that selects for cells having a heterologous nucleic acid segment. In some embodiments, methods also include a step of transforming cells with a heterologous nucleic acid segment, which may or may not be in a plasmid or other expression construct. Screening or selection can be used to identify transformed cells. In some embodiments, any one of the culturing methods and/or culturing conditions disclosed herein may be excluded as to the claimed invention.

In some embodiments, cells exposed to elevated temperatures create a cell lysate that is then exposed to cells that have not been exposed to elevated temperatures.

In particular embodiments, at least or at most 50, 55, 60, 65, 70, 75, 80, 85, 90, 95, or 100% of the cells are lysed (or any range derivable therein) within or within at least or at most 1, 2, 3, 4, 5, 6, 7, 8, 9, 10, 11, 12, 13, 14, 15, 16, 17, 18, 19, 20, 21, 22, 23, or 24 hours (or any range derivable therein) after being exposed to the elevated temperature. In further embodiments, methods also involve measuring cell lysis after the photosynthetic bacteria have been exposed to the elevated temperature.

In specific instances, cell lysis is not induced significantly by exposure to carbon dioxide or metal.

In further embodiments of methods, there is a step of evaluating transformed cells for proteolytic activity of the thermostable protease. In certain embodiments, transformed cells are evaluated for proteolytic activity of the thermostable protease after exposing cells to two different temperatures above 40° C.

A particular method is directed to inducing lysis of photosynthetic bacteria cells can include exposing the bacteria cells to an elevated temperature between about 45° C. and about 100° C., wherein the bacteria cells comprise a heterologous nucleic acid segment encoding a thermostable protease.

In one embodiment, there is a method of creating an inducible, self-destructing engineered photosynthetic bacteria that can include a) transforming cyanobacteria with a heterologous nucleic acid segment encoding at least one selectable marker and at least one thermostable protease, wherein the segment becomes integrated into the genome of the bacteria; and, b) culturing the cyanobacteria with an agent that selects for the selectable marker to identify transformed cyanobacteria; and, c) isolating cyanobacteria transformed with the heterologous nucleic acid segment. In another embodiment, it is contemplated that instead of cyanobacteria, algae or microalgae are used.

Another embodiment concerns methods for providing nutrients from cell lysate that includes a) exposing inducible, self-destructing engineered photosynthetic bacteria that can include a heterologous nucleic acid segment encoding at least one selectable marker and at least one thermostable protease to an elevated temperature between 40° C. and 100° C.; b) collecting cell lysate from the exposed and engineered bacteria; and c) providing nutrients from the cell lysate to nonexposed bacteria. The term “self-destructing” refers to the ability to undergo a process of regulated or controlled destruction of a cell.

Other embodiments include a method for generating lysate from cells that includes exposing the cells to an elevated temperature between about 45° C. and about 100° C., wherein the cells can include a heterologous nucleic acid segment encoding a thermostable protease; and, collecting the resulting cell lysate. In certain embodiments, it is specifically contemplated that the cell is cyanobacteria, other photosynthetic bacteria, fungus, yeast, algae, or microalgae.

In another particular embodiment, there are methods of extracting from a biomass that can include exposing inducible, self-destructing engineered biomass cells that can include a heterologous nucleic acid segment encoding at least one selectable marker and at least one thermostable protease to an elevated temperature between about 40° C. and about 100° C.; b) collecting cell lysate from the exposed and engineered biomass. In further embodiments, the cell lysate is subject to further processing such as purification or isolation methods.

In the context of the present invention 87 embodiments are described. In a first embodiment, an engineered photosynthetic bacteria cell containing a heterologous nucleic acid segment encoding a thermostable protease. Embodiment 2 is the engineered photosynthetic bacteria cell of embodiment 1, wherein the heterologous nucleic acid segment is integrated into the bacterial genome. Embodiment 3 is the engineered photosynthetic bacteria cell of embodiment 1 or 2, wherein the thermostable protease is from a bacteria. Embodiment 4 is the engineered photosynthetic bacteria cell of embodiment 3, wherein the thermostable protease is from a thermophilic bacteria. Embodiment 5 is the engineered photosynthetic bacteria cell of embodiment 1, wherein the thermostable protease is App (Aquifex pyrophilus), Ttp (Thermoanaerobacter tengcongensis), Tap (Thermoactinomyces sp. E79), Tfp (Thermomonospora fusca), gsp (Geobacillus stearothermaphilus F1), PFUL (Pyrococcus furiosus DSM3638), npr (Bacillus caldolytics), stp (Sulfolobus acidocaldarius), nprM (Bacillus stearothermaphilus), TtHB (Thermus thermophilus HB8), scpA (Alicyclobacillus sendaiensis), pro (Chaetomium thermophilum), bstp (Bacillus stearothermophilus), Afp (Aspergillus fumigatus WY-2), nprB (Bacillus subtilis WB30), or tau (Thermoascus aurantiacus). Embodiment 6 is the engineered photosynthetic bacteria cell of any one of embodiments 1 to 5, wherein the thermostable protease is a serine-type protease. Embodiment 7 is the engineered photosynthetic bacteria cell of any one of embodiments 1 to 5, wherein the thermostable protease is not a serine-type protease. Embodiment 8 is the engineered photosynthetic bacteria cell of any of embodiments 1 to 7, wherein the heterologous nucleic acid segment comprises a selectable or screenable marker. Embodiment 9 is the engineered photosynthetic bacteria cell of any one of embodiments 1 to 8, wherein the thermostable protease is under the transcriptional control of a constitutive promoter. Embodiment 10 is the engineered photosynthetic bacteria cell of any one of embodiments 1 to 8, wherein the thermostable protease is under the transcriptional control of an inducible promoter. Embodiment 11 is the engineered photosynthetic bacteria cell of any one of embodiments 1 to 10, wherein the thermostable protease is not from a bacteriophage. Embodiment 12 is the engineered photosynthetic bacteria cell of embodiment 1, wherein the photosynthetic bacteria is aerobic during photosynthesis. Embodiment 13 is the engineered photosynthetic bacteria cell of embodiment of 12, wherein the photosynthetic bacteria cell is cyanobacteria.

Embodiment 14 is an engineered cyanobacteria cell that includes an integrated, heterologous nucleic acid segment encoding at least one selectable marker and at least one thermostable protease under the control of a constitutive promoter. Embodiment 15 is the engineered photosynthetic bacteria cell of embodiment 1, wherein the cell is an engineered Synechocystis PCC6803 cell. Embodiment 16 is the engineered Synechocystis PCC6803 cell of embodiment 15, wherein the heterologous nucleic acid segment encodes Tap (Thermoactinomyces sp. E79). Embodiment 17 is the engineered Synechocystis PCC6803 cell of embodiment 15, wherein the heterologous nucleic acid segment encodes gsp (Geobacillus stearothermaphilus F1). Embodiment 18 is the engineered photosynthetic bacteria cell of embodiment 1, wherein the cell is an engineered Synechococcus PCC7002 cell. Embodiment 19 is the engineered Synechococcus PCC7002 cell of embodiment 18, wherein the heterologous nucleic acid segment encodes Tap (Thermoactinomyces sp. E79). Embodiment 20 is the engineered Synechococcus PCC7002 cell of embodiment 18, wherein the heterologous nucleic acid segment encodes Ttp (Thermoanaerobacter tengcongensis). Embodiment 21 is the engineered cell of any one of embodiments 15 to 20, wherein the thermostable protease is under the control of a constitutive promoter. Embodiment 22 is the engineered cell of embodiment 21, wherein the constitutive promoter is P_{trc}.

Embodiment 23 is a composition that includes a plurality of bacterial cells of any one of embodiments 1 to 22. Embodiment 24 is the composition of embodiment 23, further including bacteria media. Embodiment 25 is the composition of any one of embodiment 23 or 24, wherein the composition does not comprise more than a contaminating amount of nickel.

Embodiment 26 is a cell lysate that includes lysed photosynthetic bacteria cells and a nucleic acid segment encoding a heterologous thermostable protease.

Embodiment 27 is a method for inducing lysis of photosynthetic bacteria cells that includes exposing the bacteria cells to an elevated temperature between about 45° C. and about 100° C., wherein the bacteria cells comprise a heterologous nucleic acid segment encoding a thermostable protease. Embodiment 28 is the method of embodiment 27, wherein the bacteria cells are exposed to the elevated temperature for at least 1 hour. Embodiment 29 is the method of embodiment 28 wherein the bacteria cells are exposed to the elevated temperature between 2 hours and 48 hours. Embodiment 30 is the method of any one of Embodiments 27 to 29, wherein the bacteria cells are exposed to an elevated temperature between about 45° C. and about 80° C. Embodiment 31 is the method of embodiment 30, wherein the bacteria cells are exposed to an elevated temperature not more than about 60° C. Embodiment 32 is the method of embodiment 31, wherein the bacteria cells are exposed to solar heat. Embodiment 33 is the method of any one of embodiments 27 to 31, wherein the bacteria cells are in an enclosed container. Embodiment 34 is the method of embodiment 33, wherein the enclosed container is capable of holding 5 gallons of liquid. Embodiment 35 is the method of embodiment 34, wherein the enclosed container is capable of holding 25 gallons of liquid. Embodiment 36 is the method of embodiment 34, wherein the enclosed container is capable of holding 100 gallons of liquid. Embodiment 37 is the method of any one of embodiments 27 to 36, wherein the number of bacteria is between about 10⁸ and about 10³⁴. Embodiment 38 is the method of any one of embodiments 27 to 37, wherein the heterologous nucleic acid segment is integrated into the bacterial genome. Embodiment 39 is the method of any one of embodiments 27 to 38, wherein the thermostable protease is from a bacteria. Embodiment 40 is the method of embodiment 39, wherein the thermostable protease is from a thermophilic bacteria. Embodiment 41 is the method of any one of embodiments 28 to 40, wherein the thermostable protease is App (Aquifex pyrophilus), Ttp (Thermoanaerobacter tengcongensis), Tap (Thermoactinomyces sp. E79), Tfp (Thermomonospora fusca), gsp (Geobacillus stearothermaphilus F1), PFUL (Pyrococcus furiosus DSM3638), npr (Bacillus caldolytics), stp (Sulfolobus acidocaldarius), nprM (Bacillus stearothermaphilus), TtHB (Thermus thermophilus HB8), scpA (Alicyclobacillus sendaiensis), pro (Chaetomium thermophilum), bstp (Bacillus stearothermophilus), Afp (Aspergillus fumigatus WY-2), nprB (Bacillus subtilis WB30), or tau (Thermoascus aurantiacus). Embodiment 42 is the method of any one of Embodiments 27 to 41, wherein the thermostable protease is a serine-type protease. Embodiment 43 is the method of any one of Embodiments 27 to 41, wherein the thermostable protease is a neutral protease. Embodiment 44 is the method of any one of Embodiments 27 to 43, wherein the heterologous nucleic acid segment comprises a selectable or screenable marker. Embodiment 45 is the method of Embodiment 27 to 44, wherein the thermostable protease is under the transcriptional control of a constitutive promoter. Embodiment 46 is the method of any one of embodiments 27 to 44, wherein the thermostable protease is under the transcriptional control of an inducible promoter. Embodiment 47 is the method of any one of embodiments 27 to 46, wherein the thermostable protease is not from a bacteriophage. Embodiment 48 is the method of any one of embodiments 27 to 47, wherein the photosynthetic bacteria is aerobic during photosynthesis. Embodiment 49 is the method of embodiment of 33, wherein the photosynthetic bacteria cell is cyanobacteria. Embodiment 50 is the method of embodiment 27, wherein the photosynthetic bacteria cells are engineered Synechocystis PCC6803 cells. Embodiment 51 is the method of embodiment 50, wherein the heterologous nucleic acid segment encodes Tap (Thermoactinomyces sp. E79). Embodiment 52 is the method of embodiment 50, wherein the heterologous nucleic acid segment encodes gsp (Geobacillus stearothermaphilus F1). Embodiment 53 is the method of embodiment 27, wherein the photosynthetic bacteria cells are engineered Synechococcus PCC7002 cells. Embodiment 54 is the method of embodiment 53, wherein the heterologous nucleic acid segment encodes Tap (Thermoactinomyces sp. E79). Embodiment 55 is the method of embodiment 53, wherein the heterologous nucleic acid segment encodes Ttp (Thermoanaerobacter tengcongensis). Embodiment 56 is the method of any one of embodiments 50 to 55, wherein the thermostable protease is under the control of a constitutive promoter. Embodiment 57 is the method of embodiment 56, wherein the constitutive promoter is P_{trc}. Embodiment 58 is the method of any one of embodiments 27 to 57, further including collecting cell lysate from the photosynthetic bacterial cells. Embodiment 59 is the method of any one of embodiments 27 to 58, wherein exposed photosynthetic bacterial cells are lysed and resulting cell lysate is exposed to bacteria that has not been exposed to elevated temperatures. Embodiment 60 is the method of any one of embodiments 27 to 59, wherein at least 50% of the photosynthetic bacteria cells are lysed after being exposed to the elevated temperature. Embodiment 61 is the method of any one of embodiments 27 to 60, further including measuring cell lysis after the photosynthetic bacteria have been exposed to the elevated temperature. Embodiment 62 is the method of any one of embodiments 27 to 61, wherein cell lysis is not induced significantly by exposure to carbon dioxide or metal.

Embodiment 63 is a method of generating inducible, self-destructing engineered photosynthetic bacteria that includes culturing under growth conditions below about 40° C. bacteria including a heterologous nucleic acid segment encoding a thermostable protease.

Embodiment 64 is a method of creating an inducible, self-destructing engineered photosynthetic bacteria that includes a) transforming a photosynthetic bacteria with a heterologous nucleic acid segment encoding at least one selectable marker and at least one thermostable protease; and, b) culturing the bacteria with a selective agent to identify transformed photosynthetic bacteria. Embodiment 65 is the method of embodiment 64, wherein the heterologous nucleic acid segment becomes integrated into the bacterial genome. Embodiment 66 is the method of embodiment 64 or 65, further including culturing the transformed photosynthetic bacteria without the selective agent. Embodiment 67 is the method of any one of embodiments 64 to 66, further including evaluating transformed bacteria for proteolytic activity of the thermostable protease. Embodiment 68 is the method of embodiment 67, wherein transformed bacteria are evaluated for proteolytic activity of the thermostable protease after exposing bacteria to two different temperatures above 40° C. Embodiment 69 is the method of any one of embodiments 64 to 68, wherein the thermostable protease is from a bacteria. Embodiment 70 is the method of embodiment 69, wherein the thermostable protease is from a thermophilic bacteria. Embodiment 71 is the method of any one of embodiments 64 to 70, wherein the thermostable protease is App (Aquifex pyrophilus), Ttp (Thermoanaerobacter tengcongensis), Tap (Thermoactinomyces sp. E79), Tfp (Thermomonospora fusca), gsp (Geobacillus stearothermaphilus F1), PFUL (Pyrococcus furiosus DSM3638), npr (Bacillus caldolytics), stp (Sulfolobus acidocaldarius), nprM (Bacillus stearothermaphilus), TtHB (Thermus thermophilus HB8), scpA (Alicyclobacillus sendaiensis), pro (Chaetomium thermophilum), bstp (Bacillus stearothermophilus), Afp (Aspergillus fumigatus WY-2), nprB (Bacillus subtilis WB30), or tau (Thermoascus aurantiacus). Embodiment 72 is the method of any one of embodiments 64 to 71, wherein the thermostable protease is a serine-type protease. Embodiment 73 is the method of any one of Embodiments 64 to 71, wherein the thermostable protease is a neutral protease. Embodiment 74 is the method of any one of Embodiments 64 to 73, wherein the heterologous nucleic acid segment comprises a selectable or screenable marker. Embodiment 75 is the method of any one of Embodiments 64 to 74, wherein the thermostable protease is under the transcriptional control of a constitutive promoter. Embodiment 76 is the method of any one of Embodiments 64 to 74, wherein the thermostable protease is under the transcriptional control of an inducible promoter. Embodiment 77 is the method of any one of embodiments 64 to 76, wherein the thermostable protease is not from a bacteriophage. Embodiment 78 is the method of any one of embodiments 64 to 77, wherein the photosynthetic bacteria is aerobic during photosynthesis. Embodiment 79 is the method of embodiment of 62, wherein the photosynthetic bacteria cell is cyanobacteria.

Embodiment 80 is a method of creating an inducible, self-destructing engineered photosynthetic bacteria that includes a) transforming cyanobacteria with a heterologous nucleic acid segment encoding at least one selectable marker and at least one thermostable protease, wherein the segment becomes integrated into the genome of the bacteria; and, b) culturing the cyanobacteria with an agent that selects for the selectable marker to identify transformed cyanobacteria; and, c) isolating cyanobacteria transformed with the heterologous nucleic acid segment.

Embodiment 81 is a method of providing nutrients from cell lysate that includes: a) exposing inducible, self-destructing engineered photosynthetic bacteria including a heterologous nucleic acid segment encoding at least one selectable marker and at least one thermostable protease to an elevated temperature between 40° C. and 100° C.; b) collecting cell lysate from the exposed and engineered bacteria; and c) providing nutrients from the cell lysate to nonexposed bacteria.

Embodiment 82 is an inducible, self-destructing engineered photosynthetic cell that includes a heterologous nucleic acid segment encoding at least one selectable marker and at least one thermostable protease.

Embodiment 83 is an inducible, self-destructing engineered photosynthetic algae cell that includes a heterologous nucleic acid segment encoding at least one selectable marker and at least one thermostable protease.

Embodiment 84 is an inducible, self-destructing engineered photosynthetic cyanobacteria that includes a heterologous nucleic acid segment encoding at least one selectable marker and at least one thermostable protease.

Embodiment 85 is an inducible, self-destructing engineered yeast cell that includes a heterologous nucleic acid segment encoding at least one selectable marker and at least one thermostable protease.

Embodiment 86 is a method for generating lysate from cells that includes exposing the cells to an elevated temperature between 45° C. and 100° C., wherein the cells comprise a heterologous nucleic acid segment encoding a thermostable protease; and collecting the resulting cell lysate.

Embodiment 87 is a method for processing a biomass that includes exposing biomass cells to an elevated temperature between 45° C. and 100° C., wherein the cells comprise a heterologous nucleic acid segment encoding a thermostable protease; and, collecting the resulting cell lysate.

A “heterologous” nucleic acid segment refers to a nucleic acid region that is not found in that type of cell in nature in that location of the cell. In other words, the cell must be engineered to contain that nucleic acid segment.

The following includes definitions of various terms and phrases used throughout this specification.

The term “thermostable” means that the protease is capable of withstanding moderate heat without loss of characteristic properties, such as the ability to function as a protease at temperatures above 45° C.

The phrase “contaminating amount” means an amount that is incidental and not purposely included, such as an amount that is less than 0.1 percent by weight or by volume as compared to either the amount of cells or amount of the composition containing the cells.

The words “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

The terms “about” or “approximately” are defined as being close to as understood by one of ordinary skill in the art, and in one non-limiting embodiment the terms are defined to be within 10%, preferably within 5%, more preferably within 1%, and most preferably within 0.5%.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The engineered cyanobacteria cell of the present invention can “comprise,” “consist essentially of,” or “consist of” particular ingredients, components, compositions, etc. disclosed throughout the specification. With respect to the transitional phase “consisting essentially of,” in one non-limiting aspect, a basic and novel characteristic of the engineered cyanobacteria the present invention are their use in the manufacture of biofuel, biomass, and intracellular biological products.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1—Effect of temperature on the proteolytic activity of a protease from bacterium Thermoanaerobacter tengcongensis see for example Koma, et al., Extremophiles. 11(6):769-79, 2007.

FIG. 2—Sequence for app, a synthesized protease gene from Aquifex pyrophilus. SEQ ID NO.: 13.

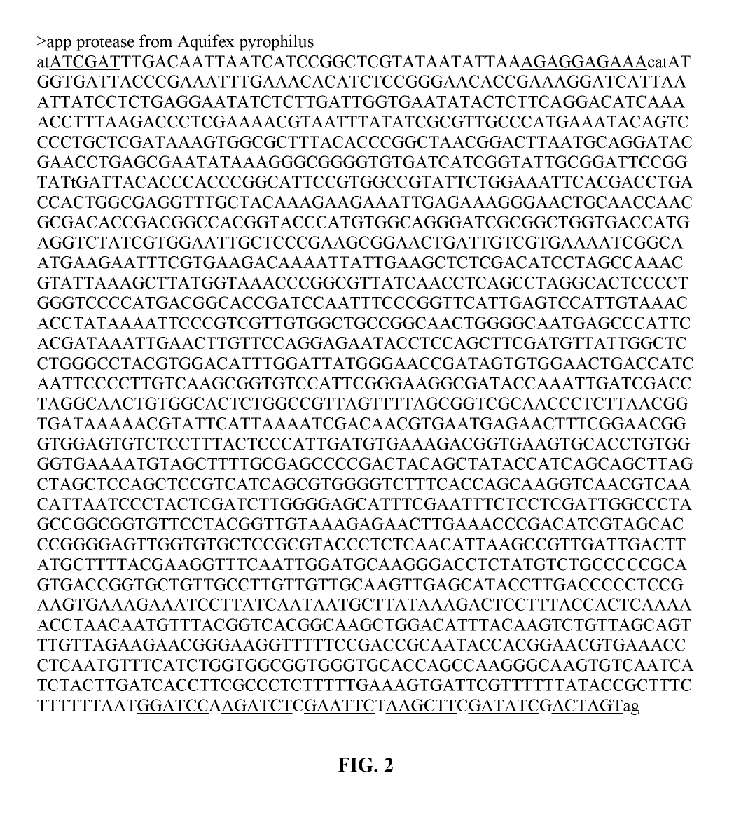

FIG. 3—Sequence for ttp, a synthesized protease gene from Thermoanaerobacter tengcongensis. SEQ ID NO.: 14.

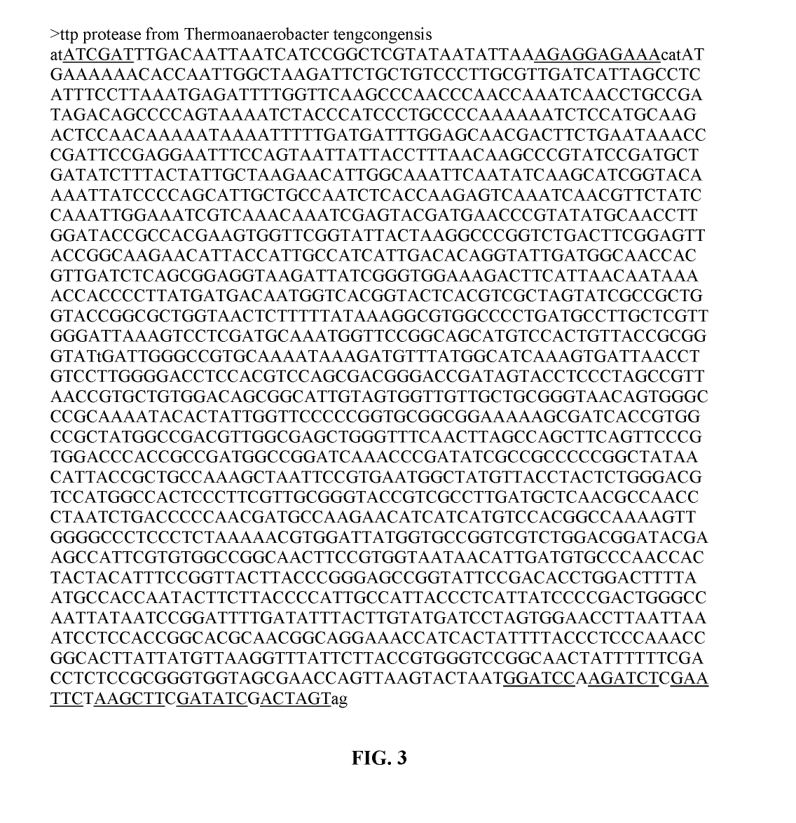

FIG. 4—Sequence for tap, a synthesized protease gene from Thermoactinomyces. SEQ ID NO.: 15.

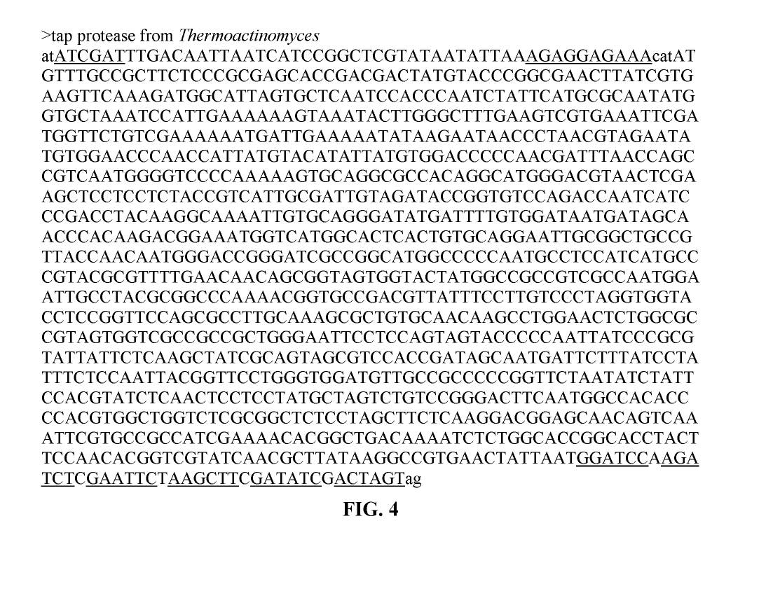

FIG. 5—Sequence for gsp, a synthesized protease gene from Geobacillus. SEQ ID NO.: 16.

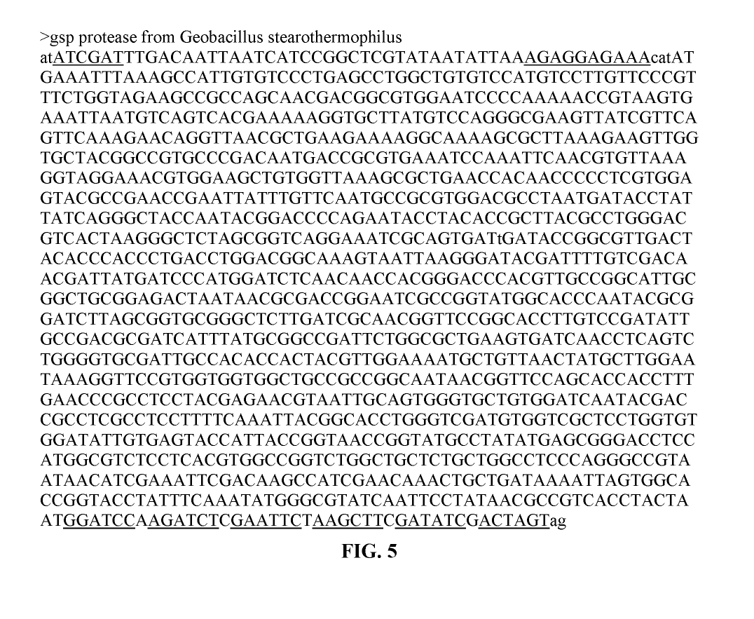

FIG. 6—Sequence for SacB KmR, a synthesized selection marker cassette. SEQ ID NO.: 17.

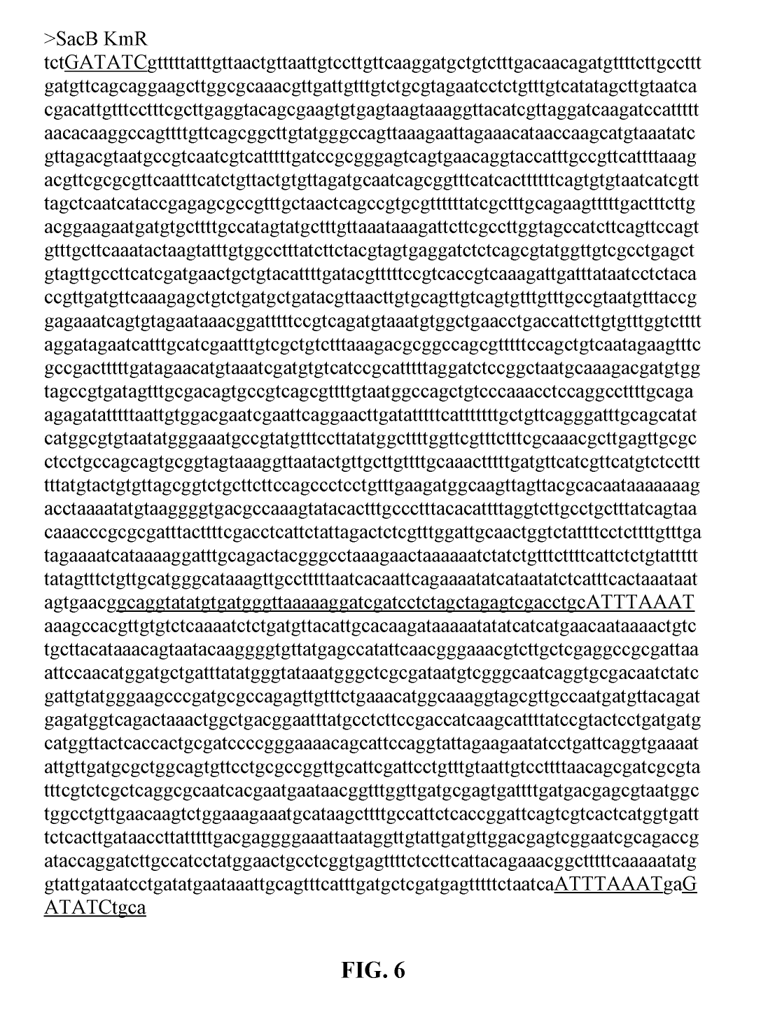

FIG. 7—The conditions of temperatures and pH for the culture inside the columns. The maximum light intensity was 1600 μmol s⁻¹ m⁻² at noon.

FIG. 8—Growth curves of Synechococcus PCC7002 and mutant cultures in outdoor bubble columns. The standard deviation between the duplication are below 5%, and not shown in the curves.

### DESCRIPTION OF ILLUSTRATIVE EMBODIMENTS

Certain embodiments are directed to the use of technology involving genetically modified cells comprising heterologous nucleotides encoding thermostable proteases. These cells may be suitable for controlled disruption of cells and/or release of intracellular biological products through temperature inducible self-destruction strategy. The technology may be particularly useful for biomass or biofuel production, as well as other applications such as feedstock or fertilizers for other organisms.

As compared to known cell disruption methods in the art that involve mechanical methods or externally added enzymes, the use of the technology in certain aspects has much better performance and higher efficiency. For example, about 100% of cells may be destructed in 24 to 48 hours with the only change of the temperature. In addition, methods and compositions described herein may be suitable for large scale production to meet commercial applications in many aspects.

Methods and compositions in certain embodiments may be described in detail below, such as thermostable proteases that may be used, nucleic acids for expressing thermostable proteases in cells, source of cells, cell culturing and disruption methods, and applications in biofuel and/or biomass production.

### I. Thermostable Proteases

In certain aspects, state-of-the-art genetic engineering technologies may be used to introduce thermostable proteases into host cells such as cyanobacteria or microalgae for biomass production. For example, thermostable protease genes were cloned into the cyanobacteria genome, thus the engineered cyanobacteria cells produced intracellular proteases at a low activity level that will not interfere with normal cell growth.

A protease (also called peptidase or proteinase) may include any enzyme that performs proteolysis, that is, begins protein catabolism by hydrolysis of the peptide bonds that link amino acids together in the polypeptide chain forming the protein. Proteases can be found in animals, plants, bacteria, archaea, funguses, and viruses.

Proteases used herein may include one or more groups of proteases based on catalytic residues, such as:

Serine proteases—using a serine alcohol;

Threonine proteases—using a threonine secondary alcohol;

Cysteine proteases—using a cysteine thiol;

Aspartate proteases—using an aspartate carboxylic acid;

Glutamic acid proteases—using a glutamate carboxylic acid;

Metalloproteases—using a metal, usually zinc.

In further aspects, proteases used herein may include one or more groups of proteases based on the optimal pH in which they are active: acid proteases, neutral proteases, or basic proteases (or alkaline proteases). In some embodiments, any one of the proteases disclosed herein may be excluded as to the claimed invention.

In certain aspects, thermostable proteases may be isolated from microorganisms that live in high temperature environment, and their activity may last long at high temperature, e.g., at least or about 45, 50, 55, 60, 65, 70, 75, 80, 85, 90, 95, 100° C., or any derivable range thereof, or any temperature above the temperature of a host cell's culture for producing desired intracellular biological products to obtain biomass and/or biofuel.

Thermostable proteases may be obtained from various organisms, such as animals, plants, bacteria, archaea, funguses, and viruses. In particular aspects, thermostable proteases may be obtained from bacteria, such as the examples in Table 2. In further aspects, thermostable proteases may be obtained from funguses, such as the examples in An et al. 2007; Nirmal & Laxman, Enzyme Research. Doi:10.1155/2014/109303, 2014, each of which is incorporated herein by reference.

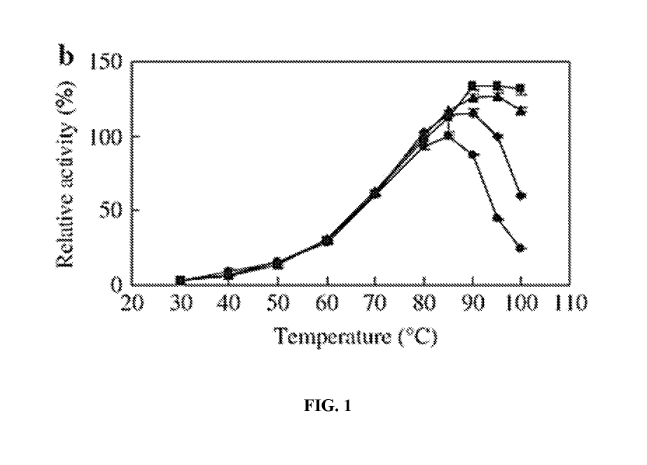

Thermostable proteases may be contemplated herein to play significant roles in industrial processing because running bioprocesses at elevated temperatures leads to higher conversion rates, an increase in the solubility and reduced risk of contamination with environmental heterotrophs. The enzyme activity of thermostable proteases increases with temperature, reaches a maximum at the optimal temperature, and then drops above the optimal temperature due to protein denaturation (FIG. 1).

The normal growth temperature of cyanobacteria is between 30-40° C., where the activity of protease is low and will not affect the cell growth. At the end of cell cultivation, cells are usually concentrated by various harvesting techniques. Then one can heat the concentrated biomass in a less volume, and the engineered cells will undergo self-destruction when proteolytic activity is increased by elevated temperatures.

Non-limiting examples of thermostable proteases are provided in Table 1, which is a library of thermostable proteases based on data reported in the literature. The proteases were ranked by their thermostability and proteolytic activity. In some embodiments, any one of the proteases disclosed herein may be excluded as to the claimed invention.

For example, synthetic genes for these thermostable proteases were designed according to the codon preference of the host cyanobacteria; Synechocystis PCC6803 and Synechococcus PCC7002 (Table 3 in Examples). The modified codon usage of each protein was back translated to create a synthetic gene, which were chemically synthesized and cloned under the control of the constitutive promoter P_{trc}. These DNA constructs were individually inserted into the host genome at the fadD site of Synechocystis and Synechococcus chromosomes by using the double cross-over recombination method.

TABLE 1

| | | | Size | Opt | Opt | | Over- | | |
|---|---|---|---|---|---|---|---|---|---|
| Rank | Name | Organism | (bp) | temp | pH | Stability | expression | Comments | Reference |
| 1 | App | Aquifex | 1857 | 95° C. | 7 | 105° C. | In E. coli | Serine- | Choi, et |
| | | pyrophilus | | | | 360 min | | type | al., J Biol |
| | | | | | | 50% | | Protease | Chem. |
| | | | | | | | | | 274(2): 881-8, |
| | | | | | | | | | 1999. |
| 2 | Ttp | Thermoanaerobacter | 1683 | 90° C. | 7 | 90° C. | In E. coli | Secreted | Koma, et |
| | | tengcongensis | | | | 143 min | | pyrolysin | al., |
| | | | | | | 50% | | | Extremophiles. |
| | | | | | | | | | 11(6): 769-79, |
| | | | | | | | | | 2007 |
| 3 | Tap | Thermoactinomyces | 1152 | 85° C. | 11.0 | 80° C. | In E. coli | alkaline | Lee, et al., |
| | | sp. E79 | | | | 10 min | | protease | Biosci |
| | | | | | | 95% | | | Biotechnol |
| | | | | | | | | | Biochem. |
| | | | | | | | | | 60(5): 840-6, |
| | | | | | | | | | 1996 |
| 4 | Tfp | Thermomonospora | 1127 | 80° C. | 9.0 | | In S. lividans | serine | Wilson |
| | | fusca | | | | | | proteinase | 1996 U.S. |
| | | | | | | | | | Pat. No. |
| | | | | | | | | | 5,705,379 |
| 5 | gsp | Geobacillus | 1591 | 80° C. | 9.0 | 80° C. | In E. coli | alkaline | Fu, et al., |
| | | stearothermophilus | | | | 240 min | | serine- | Protein |
| | | F1 | | | | 50% | | type | Expr Purif. |
| | | | | | | | | | 28(1): 63-8, |
| | | | | | | | | | 2003. |
| 6 | PFUL | Pyrococcus | 4194 | 75° C. | 7.5 | 80° C. | In B. subtilis | subtilisin | Takara, et |
| | | furiosus | | | | 180 min | | | al., J |
| | | DSM3638 | | | | 90% | | | Biochem. |
| | | | | | | | | | 124(4): 778-83, |
| | | | | | | | | | 1998 |
| 7 | npr | Bacillus | 1632 | 77° C. | 7.0 | 77° C. | In B. subtilis | neutral | Burg, J |
| | | caldolytics | | | | 60 min | | proteases | Bacterol. |
| | | | | | | 52% | | | 173: 4107-4115, |
| | | | | | | | | | 1991 |
| 8 | sip | Sulfolobus | 1020 | 75° C. | 2 | 90° C. | | Thermopsin | Lin 1990 |
| | | acidocaldarius | | | | 90 min | | | U.S. Pat. No. |
| | | | | | | 100% | | | 5,173,403 |
| 9 | nprM | Bacillus | 1656 | 70° C. | 7.5 | 90° C. | In B. subtilis | Neutral | Kubo, et |
| | | stearothermophilus | | | | 30 min | | Thermolysin | al., J Gen |
| | | | | | | 45% | | | Microbiol. |
| | | | | | | | | | 134(7): 188 |
| | | | | | | | | | 3-92, 1988 |
| 10 | TtHB | Thermus | 2385 | 70° C. | 9 | 68° C. | In E. coli | Lon | Watanabe, |
| | | thermophilus | | | | 30 min | | protease | et al., Eur J |
| | | HB8 | | | | 50% | | | Biochem. |
| | | | | | | | | | 266(3): 811-9, |
| | | | | | | | | | 1999 |
| 11 | scpA | Alicyclobacillus | 1659 | 60° C. | 3.9 | 60° C. | In E. coli | collagenolytic | Tsuruoka, |
| | | sendaiensis | | | | 60 min | | | et al., Appl |
| | | | | | | 80% | | | Environ |
| | | | | | | | | | Microbiol. |
| | | | | | | | | | 69(1): 162-9, |
| | | | | | | | | | 2003 |
| 12 | pro | Chaetomium | 2007 | 60° C. | 8.0 | 60° C. | In Pichia | serine | Li, et al., J |
| | | thermophilum | | | | 60 min | pastoris | protease | Appl |
| | | | | | | 55% | | | Microbiol. |
| | | | | | | | | | 106(2): 369-80, |
| | | | | | | | | | 2009 |
| 13 | bstp | Bacillus | ~4200 | 55° C. | 7 | 65° C. | In B. subtilis | No | Fujii, et al., |
| | | stearothermophilus | | | | 30 min | | sequence | J Bacteriol. |
| | | | | | | 80% | | | 154(2): 831-7, |
| | | | | | | | | | 1983 |
| 14 | Afp | Aspergillus | 1459 | 55° C. | 5.5 | 90° C. | In Pichiapastoris | thermostable | Wang, et |
| | | fumigatus | | | | 15 min | | phytase | al., Curr |
| | | WY-2 | | | | 50% | | | Microbiol. |
| | | | | | | | | | 55(1): 65-70, |
| | | | | | | | | | 2007 |
| 15 | nprB | Bacillus | 1614 | 50° C. | 6.6 | 65° C. | In B. subtilis | extracellular | Tran, et al., |
| | | subtilis WB30 | | | | 20 min | | | J Bacteriol. |
| | | | | | | 65% | | | 173(20): 63 |
| | | | | | | | | | 64-72, |
| | | | | | | | | | 1991 |
| 16 | tau | Thermoascus | 1482 | 50° C. | 8.0 | 70° C. | In Pichia | serine | Li, et al., J |
| | | aurantiacus | | | | 60 min | pastoris | protease | Microbiol. |
| | | | | | | 55% | | | 49(1): 121-9, |
| | | | | | | | | | 2011 |

For example, the genetically modified cells grew normally at 30° C. In the Examples, when the biomass in the culture achieved over 3 g/l, the cells were harvested and tested for self-destruction at the elevated temperature of 46° C. and above. Results in the Examples showed that the cells underwent 100% destruction at 46° C. for one day, while only 46% of wild type population showed death under the same conditions. Experiments using the cell lysate as cultivation broth for bacteria growth showed supported bacterial growth by the cyanobacterial lysate, suggesting nutritional value of the cyanobacterial lysate as a potential fermentation feedstock.

### II. Nucleic Acids for Expression of Thermostable Proteases

Methods and compositions may be used for generation of genetically modified cells to be used in commercial growth of biomass and/or biofuel, such as intracellular biological products. In certain embodiments, there are recombinant polynucleotides encoding the proteins, polypeptides, or peptides described herein for generation of genetically modified cells. Polynucleotide sequences contemplated include those encoding a thermostable protease.

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, dsRNA, siRNA, miRNA, recombinant polynucleotides, branched polynucleotides, plasmids, cosmids, phages, viruses, vectors, isolated DNA of any sequence, isolated RNA of any sequence, nucleic acid probes and primers.

The term “oligo” or “oligonucleotide” refers to polynucleotides such as deoxyribonucleic acid (DNA), and, where appropriate, ribonucleic acid (RNA). The term should also be understood to include, as equivalents, derivatives, variants and analogs of either RNA or DNA made from nucleotide analogs, and, as applicable to the embodiment being described, single (sense or antisense) and double-stranded polynucleotides. Deoxyribonucleotides include deoxyadenosine, deoxycytidine, deoxyguanosine, and deoxythymidine. For purposes of clarity, when referring herein to a nucleotide of a nucleic acid, which can be DNA or an RNA, the terms “adenosine”, “cytidine”, “guanosine”, and “thymidine” are used. It is understood that if the nucleic acid is RNA, a nucleotide having a uracil base is uridine. An oligo may be at least, about, or at most 1, 2, 3, 4, 5, 6, 7, 8, 9, 10, 20, 25, 30, 35, 40, 50, 60, 70, 80, 90, 100, 150, 200, 250, 300, 350, 400, 450, 500, 550, or 600 nucleic acids in length, or any derivable value or range thereof.

A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment that is a polynucleotide encompasses both the double-stranded form and each of two complementary single-stranded forms known or predicted to make up the double-stranded form.

Polynucleotides may include a nucleic acid molecule that either is recombinant or has been isolated free of total genomic nucleic acid. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA or synthetic), analogs thereof, or a combination thereof. Additional coding or non-coding sequences may, but need not, be present within a polynucleotide.

In this respect, the term “gene,” “polynucleotide,” or “nucleic acid” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence encoding all or a portion of such a polypeptide. It also is contemplated that a particular polypeptide may be encoded by nucleic acids containing variations having slightly different nucleic acid sequences but, nonetheless, encode the same or substantially similar protein (see above).

In particular embodiments, there are isolated nucleic acid segments and recombinant vectors incorporating nucleic acid sequences that encode a thermostable protease.

The term “recombinant” may be used in conjunction with a polypeptide or the name of a specific polypeptide, and this generally refers to a polypeptide produced from a nucleic acid molecule that has been manipulated in vitro or that is a replication product of such a molecule.

The nucleic acid segments, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length that may be limited by the ease of preparation and use in the intended recombinant nucleic acid protocol. In some cases, a nucleic acid sequence may encode a polypeptide sequence with additional heterologous coding sequences, for example to allow for purification of the polypeptide, transport, secretion, or post-translational modification.

In certain embodiments, there are polynucleotide variants having substantial identity to the sequences disclosed herein; those comprising at least 70%, 75%, 80%, 85%, 90%, 95%, 96%, 97%, 98%, or 99% or higher sequence identity, including all values and ranges there between, compared to a polynucleotide sequence provided herein using the methods described herein (e.g., BLAST analysis using standard parameters). In certain aspects, the isolated polynucleotide may comprise a nucleotide sequence encoding a polypeptide that has at least 90%, particularly 95% and above, identity (including all values and ranges there between) to an amino acid sequence described herein or known in the art, over the entire length of the sequence; or a nucleotide sequence complementary to said isolated polynucleotide.

A. Vectors

Polypeptides may be encoded by a nucleic acid molecule. The nucleic acid molecule can be in the form of a nucleic acid vector. The term “vector” is used to refer to a carrier nucleic acid molecule into which a heterologous nucleic acid sequence can be inserted for introduction into a cell where it can be replicated and expressed. A nucleic acid sequence can be “heterologous,” which means that it is in a context foreign to the cell in which the vector is being introduced or to the nucleic acid in which is incorporated, which includes a sequence homologous to a sequence in the cell or nucleic acid but in a position within the host cell or nucleic acid where it is ordinarily not found. Vectors include DNAs, RNAs, plasmids, cosmids, viruses (bacteriophage, animal viruses, and plant viruses), and artificial chromosomes (e.g., YACs). One of skill in the art would be well equipped to construct a vector through standard recombinant techniques (for example Sambrook et al., 2001; Ausubel et al., 1996, both incorporated herein by reference). Vectors may be used in a host cell to produce a thermostable protease. In some embodiments, any one of the vectors disclosed herein may be excluded as to the claimed invention.

The term “expression vector” refers to a vector containing a nucleic acid sequence coding for at least part of a gene product capable of being transcribed. In some cases, RNA molecules are then translated into a protein, polypeptide, or peptide. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described herein.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

Vectors can include a multiple cloning site (MCS), which is a nucleic acid region that contains multiple restriction enzyme sites, any of which can be used in conjunction with standard recombinant technology to digest the vector. (See Carbonelli et al., 1999, Levenson et al., 1998, and Cocea, 1997, incorporated herein by reference.)

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression. (See Chandler et al., 1997, incorporated herein by reference.)

The vectors or constructs may comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels. In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to be more stable and are translated more efficiently.

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

B. Promoters

Vectors can also comprise other components or functionalities that further modulate gene delivery and/or gene expression, or that otherwise provide beneficial properties to the targeted cells. Such other components include, for example, components that influence binding or targeting to cells; components that influence uptake of the vector nucleic acid by the cell; components that influence localization of the polynucleotide within the cell after uptake (such as agents mediating nuclear localization); and components that influence expression of the polynucleotide. In some embodiments, any one of these other components disclosed herein may be excluded as to the claimed invention.

A “promoter” is a control sequence. The promoter may be a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and expression of that sequence. A promoter may or may not be used in conjunction with an “enhancer,” which refers to a cis-acting regulatory sequence involved in the transcriptional activation of a nucleic acid sequence.

In some aspects, a particular promoter that is employed to control the expression of a peptide or protein encoding polynucleotide is not believed to be critical, so long as it is capable of expressing the polynucleotide in a targeted host cell, particularly a bacterial cell. In certain aspects, such a promoter might include either a bacterial, human or viral promoter. A specific initiation signal also may be used for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals.

In exemplary embodiments, the disclosed polynucleotides are operably connected to a promoter in the polynucleotide construct. As is understood in the art, a promoter may be a segment of DNA which acts as a controlling element in the expression of that gene. In one embodiment, the promoter is a native Anabaena promoter. For example, the promoter may be an Anabaena Pnir promoter such as the one described in Desplancq, D2005, Combining inducible protein overexpression with NMR-grade triple isotope labeling in the cyanobacterium Anabaena sp. PCC 7120. Biotechniques. 39:405-11 or one having sequence identity of about 76%, 80%, 85%, at least about 90%, and at least about 95%, 96%, 97%, 98% or 99% to the disclosed promoter sequence. The promoter may also be an Anabaena psbA promoter, PrbcL promoter and/or E. coli Ptac promoter (Elhai, J. 1993. Strong and regulated promoters in the cyanobacterium Anabaena PCC 7120. FEMS Microbiol Lett. 114(2):179-84) or one having sequence identity of about 76%, 80%, 85%, at least about 90%, and at least about 95%, 96%, 97%, 98% or 99% to any of the disclosed promoter sequences. In some embodiments, the promoter is a combined dual promoter, i.e. a promoter containing more than one of the above or known in the art.

A promoter may be a control sequence that is a region of a nucleic acid sequence at which initiation and rate of transcription are controlled. It may contain genetic elements at which regulatory proteins and molecules may bind, such as RNA polymerase and other transcription factors, to initiate the specific transcription a nucleic acid sequence.

A promoter may comprise a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30-110 bp upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently may be flexible, so that promoter function is preserved when elements are inverted or moved relative to one another. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the β-lactamase (penicillinase), lactose and tryptophan (trp) promoter systems.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al. 1989, incorporated herein by reference). The promoters employed may be constitutive, cell type-specific, tissue-specific, inducible, and/or useful under the appropriate conditions to direct high level expression of the introduced DNA segment, such as is advantageous in the large-scale production of recombinant proteins and/or peptides. The promoter may be heterologous or endogenous. In some embodiments, any one of the promoters disclosed herein may be excluded as to the claimed invention.

Certain nucleic acid constructs may comprise an inducible promoter, or may be a constitutive promoter. Non-limiting examples of inducible promoters may include, but are not limited to, those induced by expression of an exogenous protein (e.g., T7 RNA polymerase, SP6 RNA polymerase), by the presence of a small molecule (e.g., IPTG, galactose, tetracycline, steroid hormone, abscisic acid), by metals or metal ions (e.g., copper, zinc, cadmium, nickel), and by environmental factors (e.g., heat, cold, stress). In each of the above embodiments, the inducible promoter is preferably tightly regulated such that in the absence of induction, substantially no transcription is initiated through the promoter. Additionally, induction of the promoter of interest should not typically alter transcription through other promoters. Also, generally speaking, the compound or condition that induces an inducible promoter should not be naturally present in the organism or environment where expression is sought.

Non-limiting examples of constitutive promoters may include constitutive promoters from Gram negative bacteria or a Gram negative bacteriophage. For instance, promoters from highly expressed Gram negative gene products may be used, such as the promoter for Lpp, OmpA, rRNA, and hbosomal proteins. Alternatively, regulatable promoters may be used in a strain that lacks the regulatory protein for that promoter. For instance Ptac, Ptac, and Ptrc may be used as constitutive promoters in strains that lack Lacl. Similarly, P22 PR and PL may be used in strains that lack the P22 C2 repressor protein, and λ PR and PL may be used in strains that lack the λ C1 repressor protein. In one embodiment, the constitutive promoter is from a bacteriophage. In another embodiment, the constitutive promoter is from a Salmonella bacteriophage. In yet another embodiment, the constitutive promoter is from a cyanophage. In some embodiments, the constitutive promoter is a Synechocystis promoter. For instance, the constitutive promoter may be the PpSbAit promoter.

In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202 and 5,928,906, each incorporated herein by reference).

C. Selectable or Screenable Markers

A selectable marker may be a gene introduced into a cell or to cells in culture, that confers a trait suitable for artificial selection or screen. Selectable markers may include a type of reporter gene used in laboratory microbiology, molecular biology, and genetic engineering to indicate the success of a transfection or other procedure meant to introduce foreign DNA into a cell. Selectable markers are often antibiotic resistance genes; cells that have been subjected to a procedure to introduce foreign DNA are grown on a medium containing an antibiotic, and those cells that can grow have successfully taken up and expressed the introduced genetic material. Examples of selectable markers include: the Abicr gene or Neo gene from Tn5, which confers antibiotic resistance to geneticin.

Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, blasticidin, DHFR, GPT, zeocin and histidinol are useful selectable markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes such as chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art. In some embodiments, any one of these selectable and/or screenable markers disclosed herein may be excluded as to the claimed invention.

A screenable marker may comprise a reporter gene, which allows the researcher to distinguish between wanted and unwanted cells. Examples of such reporters include genes encoding cell surface proteins (e.g., CD4, HA epitope), fluorescent proteins, antigenic determinants and enzymes (e.g., β-galactosidase). The vector containing cells may be isolated, e.g., by FACS using fluorescently-tagged antibodies to the cell surface protein or substrates that can be converted to fluorescent products by a vector encoded enzyme.

In specific embodiments, the reporter gene is a fluorescent protein. A broad range of fluorescent protein genetic variants have been developed that feature fluorescence emission spectral profiles spanning almost the entire visible light spectrum (see Table 2 for non-limiting examples of fluorescent protein properties). Mutagenesis efforts in the original Aequorea victoria jellyfish green fluorescent protein have resulted in new fluorescent probes that range in color from blue to yellow, and are some of the most widely used in vivo reporter molecules in biological research. Longer wavelength fluorescent proteins, emitting in the orange and red spectral regions, have been developed from the marine anemone, Discosoma striata, and reef corals belonging to the class Anthozoa. Still other species have been mined to produce similar proteins having cyan, green, yellow, orange, and deep red fluorescence emission.

TABLE 2

| | | | | | | Relative |
|---|---|---|---|---|---|---|
| | Excitation | Emission | Molar | | | Brightness |
| Protein | Maximum | Maximum | Extinction | Quantum | in vivo | (% of |
| (Acronym) | (nm) | (nm) | Coefficient | Yield | Structure | EGFP) |
| GFP (wt) | 395/475 | 509 | 21,000 | 0.77 | Monomer* | 48 |

Green Fluorescent Proteins

| | | | | | | |
|---|---|---|---|---|---|---|
| EGFP | 484 | 507 | 56,000 | 0.60 | Monomer* | 100 |
| AcGFP | 480 | 505 | 50,000 | 0.55 | Monomer* | 82 |
| TurboGFP | 482 | 502 | 70,000 | 0.53 | Monomer* | 110 |
| Emerald | 487 | 509 | 57,500 | 0.68 | Monomer* | 116 |
| Azami Green | 492 | 505 | 55,000 | 0.74 | Monomer | 121 |
| ZsGreen | 493 | 505 | 43,000 | 0.91 | Tetramer | 117 |

Blue Fluorescent Proteins

| | | | | | | |
|---|---|---|---|---|---|---|
| EBFP | 383 | 445 | 29,000 | 0.31 | Monomer* | 27 |
| Sapphire | 399 | 511 | 29,000 | 0.64 | Monomer* | 55 |
| T-Sapphire | 399 | 511 | 44,000 | 0.60 | Monomer* | 79 |

Cyan Fluorescent Proteins

| | | | | | | |
|---|---|---|---|---|---|---|
| ECFP | 439 | 476 | 32,500 | 0.40 | Monomer* | 39 |
| mCFP | 433 | 475 | 32,500 | 0.40 | Monomer | 39 |
| Cerulean | 433 | 475 | 43,000 | 0.62 | Monomer* | 79 |
| CyPet | 435 | 477 | 35,000 | 0.51 | Monomer* | 53 |
| AmCyan1 | 458 | 489 | 44,000 | 0.24 | Tetramer | 31 |
| Midori-Ishi Cyan | 472 | 495 | 27,300 | 0.90 | Dimer | 73 |
| mTFP1 (Teal) | 462 | 492 | 64,000 | 0.85 | Monomer | 162 |

Yellow Fluorescent Proteins

| | | | | | | |
|---|---|---|---|---|---|---|
| EYFP | 514 | 527 | 83,400 | 0.61 | Monomer* | 151 |
| Topaz | 514 | 527 | 94,500 | 0.60 | Monomer* | 169 |
| Venus | 515 | 528 | 92,200 | 0.57 | Monomer* | 156 |
| mCitrine | 516 | 529 | 77,000 | 0.76 | Monomer | 174 |
| YPet | 517 | 530 | 104,000 | 0.77 | Monomer* | 238 |
| PhiYFP | 525 | 537 | 124,000 | 0.39 | Monomer* | 144 |
| ZsYellow1 | 529 | 539 | 20,200 | 0.42 | Tetramer | 25 |
| mBanana | 540 | 553 | 6,000 | 0.7 | Monomer | 13 |

Orange and Red Fluorescent Proteins

| | | | | | | |
|---|---|---|---|---|---|---|
| Kusabira Orange | 548 | 559 | 51,600 | 0.60 | Monomer | 92 |
| mOrange | 548 | 562 | 71,000 | 0.69 | Monomer | 146 |
| dTomato | 554 | 581 | 69,000 | 0.69 | Dimer | 142 |
| dTomato-Tandem | 554 | 581 | 138,000 | 0.69 | Monomer | 283 |
| DsRed | 558 | 583 | 75,000 | 0.79 | Tetramer | 176 |
| DsRed2 | 563 | 582 | 43,800 | 0.55 | Tetramer | 72 |
| DsRed-Express | 555 | 584 | 38,000 | 0.51 | Tetramer | 58 |
| (T1) | | | | | | |
| DsRed-Monomer | 556 | 586 | 35,000 | 0.10 | Monomer | 10 |
| mTangerine | 568 | 585 | 38,000 | 0.30 | Monomer | 34 |
| mStrawberry | 574 | 596 | 90,000 | 0.29 | Monomer | 78 |
| AsRed2 | 576 | 592 | 56,200 | 0.05 | Tetramer | 8 |
| mRFP1 | 584 | 607 | 50,000 | 0.25 | Monomer | 37 |
| JRed | 584 | 610 | 44,000 | 0.20 | Dimer | 26 |
| mCherry | 587 | 610 | 72,000 | 0.22 | Monomer | 47 |
| HcRed1 | 588 | 618 | 20,000 | 0.015 | Dimer | 1 |
| mRaspberry | 598 | 625 | 86,000 | 0.15 | Monomer | 38 |
| HcRed-Tandem | 590 | 637 | 160,000 | 0.04 | Monomer | 19 |
| mPlum | 590 | 649 | 41,000 | 0.10 | Monomer | 12 |
| AQ143 | 595 | 655 | 90,000 | 0.04 | Tetramer | 11 |

*Weak Dimer

D. Methods of Gene Transfer

Suitable methods for nucleic acid delivery to effect expression of compositions are believed to include virtually any method by which a nucleic acid (e.g., DNA, including viral and nonviral vectors) can be introduced into a cell, a tissue or an organism, as described herein or as would be known to one of ordinary skill in the art. Such methods include, but are not limited to, direct delivery of DNA such as by injection (See, for example, U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (See, for example, Harland and Weintraub, 1985; U.S. Pat. No. 5,789,215, incorporated herein by reference); by electroporation (See, for example, U.S. Pat. No. 5,384,253, incorporated herein by reference); by calcium phosphate precipitation (See, for example, Graham and Van Der Eb, 1973; Chen and Okayama, 1987; Rippe et al., 1990); by using DEAE dextran followed by polyethylene glycol (Gopal, 1985); by direct sonic loading (See, for example, Fechheimer et al., 1987); by liposome mediated transfection (See, for example, Nicolau and Sene, 1982; Fraley et al., 1979; Nicolau et al., 1987; Wong et al., 1980; Kaneda et al., 1989; Kato et al., 1991); by microprojectile bombardment (See, for example, PCT Application Nos. WO 94/09699 and 95/06128; U.S. Pat. Nos. 5,610,042; 5,322,783, 5,563,055, 5,550,318, 5,538,877 and 5,538,880, and each incorporated herein by reference); by agitation with silicon carbide fibers (See, for example, Kaeppler et al., 1990; U.S. Pat. Nos. 5,302,523 and 5,464,765, each incorporated herein by reference); by Agrobacterium mediated transformation (See, for example, U.S. Pat. Nos. 5,591,616 and 5,563,055, each incorporated herein by reference); or by PEG mediated transformation of protoplasts (See, for example, Omirulleh et al., 1993; U.S. Pat. Nos. 4,684,611 and 4,952,500, each incorporated herein by reference); by desiccation/inhibition mediated DNA uptake (see, for example, Potrykus et al., 1985). Through the application of techniques such as these, organelle(s), cell(s), tissue(s) or organism(s) may be stably or transiently transformed. In some embodiments, any one of these methods for nucleic acid delivery disclosed herein may be excluded as to the claimed invention.

In particular embodiments, a recombinant expression construct for transformation of a host cell and subsequent integration of the gene(s) of interest is prepared by first isolating the constituent polynucleotide sequences. In some embodiments, the gene(s) of interest are homologously integrated into the host cell genome. In other embodiments, the genes are non-homologously integrated into the host cell genome.

Constructs containing polynucleotides may be introduced into the host cell using a standard protocol, such as the one set out in, for example, Golden, et al., Methods Enzymol 153: 215-231, 1987 and in Golden & Sherman, J. Bacteriol. 158:36, 1984, both of which are incorporated herein by reference. Any of the well-known procedures for introducing heterologous polynucleotide sequences into host cells can be used. In certain embodiments, only a single copy of the heterologous polynucleotide is introduced. In other embodiments, more than a single copy, such as two copies, three copies or more than three copies of the heterologous polynucleotide is introduced. As is understood by the skilled artisan, multiple copies of heterologous polynucleotides may be on a single construct or on more than one construct.

In some embodiments, the gene of interest is transiently introduced into the host cell through use of a plasmid or shuttle vector. In other embodiments, the gene of interest is permanently introduced into the chromosome of the host cell. Chromosomal integration techniques are known to the skilled artisan and have been described in, for example, Zhou & Wolk, J Bacteriol., 184(9):2529-2532, 2002. Briefly, the gene of interest is fused to a promoter and then subcloned into an integration vector. This construct may be introduced into the host cell for double homologous recombination at specific loci on the host cell chromosome.

E. Linkers

Any polypeptides expressed from heterologous nucleotides herein can be truncated and replaced by short linkers or they can be conjugated by linkers. In some embodiments, the polypeptide include one or more peptide linkers. For example, a linker consists of from 2 to 25 amino acids. In further aspects, it is from 2 to 15 amino acids in length, although in certain circumstances, it can be only one, such as a single glycine residue.

In one embodiment, a nucleic acid molecule, in which polynucleotide encoding a first polypeptide such as a thermostable protease is genetically fused with polynucleotide encoding a second polypeptide such as another thermostable protease or a selectable or screenable marker, is expressed in a host cell so that the first attachment site and the second attachment site are linked through a peptide bond. In this case, the two polypeptides may be linked through a peptide bond. Relating to this embodiment, the first attachment site and/or the second attachment site may be genetically modified from the original polypeptide. For example, the first attachment site is modified from the polypeptide so that through a linker peptide including SG, GS, SGG, GGS and SGSG, the first polypeptide is conjugated with the second one.

When the polypeptide are chemically conjugated with another polypeptide, the first attachment site and the second attachment site may be linked through a chemical cross-linker which is a chemical compound.

Examples of the cross-linker include, but are not limited to, SMPH, Sulfo-MBS, Sulfo-EMCS, Sulfo-GMBS, Sulfo-SIAB, Sulfo-SMPB, Sulfo-SMCC, SVSB, SIA and other cross-linkers available from the Pierce Chemical Company.

If desired, the peptides of interest may be joined via a biologically-releasable bond, such as a selectively-cleavable linker or amino acid sequence. For example, peptide linkers that include a cleavage site for an enzyme preferentially located or active within a tumor environment are contemplated. Exemplary forms of such peptide linkers are those that are cleaved by urokinase, plasmin, thrombin, Factor IXa, Factor Xa, or a metallaproteinase, such as collagenase, gelatinase, or stromelysin.

Additionally, while numerous types of disulfide-bond containing linkers are known which can successfully be employed to conjugate moieties, certain linkers may be chosen over other linkers, based on differing pharmacologic characteristics and capabilities. For example, linkers that contain a disulfide bond that is sterically “hindered” are to be preferred, due to their greater stability in vivo, thus preventing release of the moiety prior to binding at the site of action.

Additionally, any other linking/coupling agents and/or mechanisms known to those of skill in the art can be used to combine peptides, components or agents of interest, such as, for example, antibody-antigen interaction, avidin biotin linkages, amide linkages, ester linkages, thioester linkages, ether linkages, thioether linkages, phosphoester linkages, phosphoramide linkages, anhydride linkages, disulfide linkages, ionic and hydrophobic interactions, bispecific antibodies and antibody fragments, or combinations thereof.

Cross-linking reagents may be used to form molecular bridges that tie together functional groups of two different molecules, e.g., a thermostable protease and a selectable or screenable marker. In certain aspects, it is contemplated that dimers or multimers of the same thermostable protease can be made or that heteromeric complexes comprised of different thermostable proteases can be created. To link two different compounds or peptides in a step-wise manner, hetero-bifunctional cross-linkers can be used to eliminate unwanted homopolymer formation.

In some embodiments, any one of the linking/coupling agents and/or mechanisms disclosed herein may be excluded as to the claimed invention.

### III. Sources of Cells for Expressing Thermostable Proteases

Any cells may be used in certain aspects for producing desired biological products, including, but not limited to, bacteria, yeasts and/or algae such as microalgae. In certain embodiments, photosynthetic cells may be used, including, but not limited to, cells of eukaryotic plants and algae, as well as prokaryotic cyanobacteria, green-sulfur bacteria, green non-sulfur bacteria, purple sulfur bacteria, and purple non-sulfur bacteria. In some embodiments, any one of the cells disclosed herein may be excluded as to the claimed invention.

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector or expressing a heterologous gene encoded by a vector. A host cell can, and has been, used as a recipient for vectors or viruses. A host cell may be “transfected” or “transformed,” which refers to a process by which exogenous nucleic acid, such as a recombinant protein-encoding sequence, is transferred or introduced into the host cell. A transformed cell includes the primary subject cell and its progeny.

In particular aspects, host cell used herein may be any bacteria that can produce desired biological products, such as photosynthetic bacteria or non-photosynthetic bacteria. In further aspects, the photosynthetic bacteria may be cyanobacteria or microalgae.

In certain aspects, the cells may be microphytes or microalgae that are microscopic algae, typically found in freshwater and marine systems. They are unicellular species which exist individually, or in chains or groups. Depending on the species, their sizes can range from a few micrometers (μm) to a few hundreds of micrometers. Unlike higher plants, microalgae do not have roots, stems and leaves. Microalgae, capable of performing photosynthesis, are important for life on earth; they produce approximately half of the atmospheric oxygen and use simultaneously the greenhouse gas carbon dioxide to grow photoautotrophically.

Microalgae may refer to an eukaryotic microbial organism that contains a chloroplast, and optionally that is capable of performing photosynthesis, or a prokaryotic microbial organism capable of performing photosynthesis. Microalgae may include obligate photoautotrophs, which cannot metabolize a fixed carbon source as energy, as well as heterotrophs, which can live solely off of a fixed carbon source. Microalgae can also refer to unicellular organisms that separate from sister cells shortly after cell division, such as Chlamydomonas, and can also refer to microbes such as, for example, Volvox, which is a simple multicellular photosynthetic microbe of two distinct cell types. Microalgae can also refer to cells such as Chlorella, Parachlorella and Dunaliella. Microalgae may also include other microbial photosynthetic organisms that exhibit cell-cell adhesion, such as Agmenellum, Anabaena, and Pyrobotrys. “Microalgae” may also include obligate heterotrophic microorganisms that have lost the ability to perform photosynthesis, such as certain dinoflagellate algae species.

In further aspects, the cells may be cyanobacteria, such as photosynthetic bacteria that produce organic matters using CO₂, sunlight and water. Cyanobacteria are being considered as a source of renewable feedstock for chemicals by many companies. In this process, growing algal biomass in ponds or photo-bioreactors is a key requirement with good process control. After achieving an ascertained biomass, the algal biomass needs to be harvested, disrupted by cell lysis and the target product extracted.

In certain embodiments, photosynthetic cells or cells that may be used herein may be one or more cells of an algae and/or cyanobacterial organism selected from Acanthoceras, Acanthococcus, Acaryochloris, Achnanthes, Achnanthidium, Actinastrum, Actinochloris, Actinocyclus, Actinotaenium, Amphichrysis, Amphidinium, Amphikrikos, Amphipleura, Amphiprora, Amphithrix, Amphora, Anabaena, Anabaenopsis, Aneumastus, Ankistrodesmus, Ankyra, Anomoeoneis, Apatococcus, Aphanizomenon, Aphanocapsa, Aphanochaete, Aphanothece, Apiocystis, Apistonema, Arthrodesmus, Artherospira, Ascochloris, Asterionella, Asterococcus, Audouinella, Aulacoseira, Bacillaria, Balbiania, Bambusina, Bangia, Basichlamys, Batrachospermum, Binuclearia, Bitrichia, Blidingia, Botrdiopsis, Botrydium, Botryococcus, Botryosphaerella, Brachiomonas, Brachysira, Brachytrichia, Brebissonia, Bulbochaete, Bumilleria, Bumilleriopsis, Caloneis, Calothrix, Campylodiscus, Capsosiphon, Carteria, Catena, Cavinula, Centritr actus, Centronella, Ceratium, Chaetoceros, Chaetochloris, Chaetomorpha, Chaetonella, Chaetonema, Chaetopeltis, Chaetophora, Chaetosphaeridium, Chamaesiphon, Chara, Characiochloris, Characiopsis, Characium, Charales, Chilomonas, Chlainomonas, Chlamydoblepharis, Chlamydocapsa, Chlamydomonas, Chlamydomonopsis, Chlamydomyxa, Chlamydonephris, Chlorangiella, Chlorangiopsis, Chlorella, Chlorobotrys, Chlorobrachis, Chlorochytrium, Chlorococcum, Chlorogloea, Chlorogloeopsis, Chlorogonium, Chlorolobion, Chloromonas, Chlorophysema, Chlorophyta, Chlorosaccus, Chlorosarcina, Choricystis, Chromophyton, Chromulina, Chroococcidiopsis, Chroococcus, Chroodactylon, Chroomonas, Chroothece, Chrysamoeba, Chrysapsis, Chrysidiastrum, Chrysocapsa, Chrysocapsella, Chrysochaete, Chrysochromulina, Chrysococcus, Chrysocrinus, Chrysolepidomonas, Chrysolykos, Chrysonebula, Chrysophyta, Chrysopyxis, Chrysosaccus, Chrysophaerella, Chrysostephanosphaera, Clodophora, Clastidium, Closteriopsis, Closterium, Coccomyxa, Cocconeis, Coelastrella, Coelastrum, Coelosphaerium, Coenochloris, Coenococcus, Coenocystis, Colacium, Coleochaete, Collodictyon, Compsogonopsis, Compsopogon, Conjugatophyta, Conochaete, Coronastrum, Cosmarium, Cosmioneis, Cosmocladium, Crateriportula, Craticula, Crinalium, Crucigenia, Crucigeniella, Cryptoaulax, Cryptomonas, Cryptophyta, Ctenophora, Cyanodictyon, Cyanonephron, Cyanophora, Cyanophyta, Cyanothece, Cyanothomonas, Cyclonexis, Cyclostephanos, Cyclotella, Cylindrocapsa, Cylindrocystis, Cylindrospermum, Cylindrotheca, Cymatopleura, Cymbella, Cymbellonitzschia, Cystodinium Dactylococcopsis, Debarya, Denticula, Dermatochrysis, Dermocarpa, Dermocarpella, Desmatr actum, Desmidium, Desmococcus, Desmonema, Desmosiphon, Diacanthos, Diacronema, Diadesmis, Diatoma, Diatomella, Dicellula, Dichothrix, Dichotomococcus, Dicranochaete, Dictyochloris, Dictyococcus, Dictyosphaerium, Didymocystis, Didymogenes, Didymosphenia, Dilabifilum, Dimorphococcus, Dinobryon, Dinococcus, Diplochloris, Diploneis, Diplostauron, Distrionella, Docidium, Draparnaldia, Dunaliella, Dysmorphococcus, Ecballocystis, Elakatothrix, Ellerbeckia, Encyonema, Enteromorpha, Entocladia, Entomoneis, Entophysalis, Epichrysis, Epipyxis, Epithemia, Eremosphaera, Euastropsis, Euastrum, Eucapsis, Eucocconeis, Eudorina, Euglena, Euglenophyta, Eunotia, Eustigmatophyta, Eutreptia, Fallacia, Fischerella, Fragilaria, Fragilariforma, Franceia, Frustulia, Curcilla, Geminella, Genicularia, Glaucocystis, Glaucophyta, Glenodiniopsis, Glenodinium, Gloeocapsa, Gloeochaete, Gloeochrysis, Gloeococcus, Gloeocystis, Gloeodendron, Gloeomonas, Gloeoplax, Gloeothece, Gloeotila, Gloeotrichia, Gloiodictyon, Golenkinia, Golenkiniopsis, Gomontia, Gompho cymbella, Gomphonema, Gomphosphaeria, Gonatozygon, Gongrosia, Gongrosira, Goniochloris, Gonium, Gonyostomum, Granulochloris, Granulocy stop sis, Groenbladia, Gymnodinium, Gymnozyga, Gyrosigma, Haemato coccus, Hafniomonas, Hallassia, Hammatoidea, Hannaea, Hantzschia, Hapalosiphon, Haplotaenium, Haptophyta, Haslea, Hemidinium, Hemitoma, Heribaudiella, Heteromastix, Heterothrix, Hibberdia, Hildenbrandia, Hillea, Holopedium, Homoeothrix, Hormanthonema, Hormotila, Hyalobrachion, Hyalocardium, Hyalodiscus, Hyalogonium, Hyalotheca, Hydrianum, Hydrococcus, Hydrocoleum, Hydrocoryne, Hydrodictyon, Hydrosera, Hydrurus, Hyella, Hymenomonas, Isthmochloron, Johannesbaptistia, Juranyiella, Karayevia, Kathablepharis, Katodinium, Kephyrion, Keratococcus, Kirchneriella, Klebsormidium, Kolbesia, Koliella, Komarekia, Korshikoviella, Kraskella, Lagerheimia, Lagynion, Lamprothamnium, Lemanea, Lepocinclis, Leptosira, Lobococcus, Lobocystis, Lobomonas, Luticola, Lyngbya, Malleochloris, Mallomonas, Mantoniella, Marssoniella, Martyana, Mastigocoleus, Gastogloia, Melosira, Merismopedia, Mesostigma, Mesotaenium, Micractinium, Micrasterias, Microchaete, Microcoleus, Microcystis, Microglena, Micromonas, Microspora, Microthamnion, Mischococcus, Monochrysis, Monodus, Monomastix, Monoraphidium, Monostroma, Mougeotia, Mougeotiopsis, Myochloris, Myromecia, Myxosarcina, Naegeliella, Nannochloris, Nautococcus, Navicula, Neglectella, Neidium, Nephroclamys, Nephrocytium, Nephrodiella, Nephroselmis, Netrium, Nitella, Nitellopsis, Nitzschia, Nodularia, Nostoc, Ochromonas, Oedogonium, Oligochaetophora, Onychonema, Oocardium, Oocystis, Opephora, Ophiocytium, Orthoseira, Oscillatoria, Oxyneis, Pachycladella, Palmella, Palmodictyon, Pnadorina, Pannus, Paralia, Pascherina, Paulschulzia, Pediastrum, Pedinella, Pedinomonas, Pedinopera, Pelagodictyon, Penium, Peranema, Peridiniopsis, Peridinium, Peronia, Petroneis, Phacotus, Phacus, Phaeaster, Phaeodermatium, Phaeophyta, Phaeosphaera, Phaeothamnion, Phormidium, Phycopeltis, Phyllariochloris, Phyllocardium, Phyllomitas, Pinnularia, Pitophora, Placoneis, Planctonema, Planktosphaeria, Planothidium, Plectonema, Pleodorina, Pleurastrum, Pleurocapsa, Pleurocladia, Pleurodiscus, Pleurosigma, Pleurosira, Pleurotaenium, Pocillomonas, Podohedra, Polyblepharides, Polychaetophora, Polyedriella, Polyedriopsis, Polygoniochloris, Polyepidomonas, Polytaenia, Polytoma, Polytomella, Porphyridium, Posteriochromonas, Prasinochloris, Prasinocladus, Prasinophyta, Prasiola, Prochlorphyta, Prochlorothrix, Protoderma, Protosiphon, Provasoliella, Prymnesium, Psammodictyon, Psammothidium, Pseudanabaena, Pseudenoclonium, Psuedocarteria, Pseudochate, Pseudocharacium, Pseudococcomyxa, Pseudodictyosphaerium, Pseudokephyrion, Pseudoncobyrsa, Pseudoquadrigula, Pseudosphaerocystis, Pseudostaurastrum, Pseudostaurosira, Pseudotetrastrum, Pteromonas, Punctastruata, Pyramichlamys, Pyramimonas, Pyrrophyta, Quadrichloris, Quadricoccus, Quadrigula, Radiococcus, Radiofilum, Raphidiopsis, Raphidocelis, Raphidonema, Raphidophyta, Peimeria, Rhabdoderma, Rhabdomonas, Rhizoclonium, Rhodomonas, Rhodophyta, Rhoicosphenia, Rhopalodia, Rivularia, Rosenvingiella, Rossithidium, Roya, Scenedesmus, Scherffelia, Schizochlamydella, Schizochlamys, Schizomeris, Schizothrix, Schroederia, Scolioneis, Scotiella, Scotiellopsis, Scourfieldia, Scytonema, Selenastrum, Selenochloris, Sellaphora, Semiorbis, Siderocelis, Diderocystopsis, Dimonsenia, Siphononema, Sirocladium, Sirogonium, Skeletonema, Sorastrum, Spermatozopsis, Sphaerellocystis, Sphaerellopsis, Sphaerodinium, Sphaeroplea, Sphaerozosma, Spiniferomonas, Spiwgyra, Spirotaenia, Spirulina, Spondylomorum, Spondylosium, Sporotetras, Spumella, Staurastrum, Stauerodesmus, Stauroneis, Staurosira, Staurosirella, Stenopterobia, Stephanocostis, Stephano'discus, Stephanoporos, Stephanosphaera, Stichococcus, Stichogloea, Stigeoclonium, Stigonema, Stipitococcus, Stokesiella, Strombomonas, Stylo chrysalis, Stylodinium, Styloyxis, Stylosphaeridium, Surirella, Sykidion, Symploca, Synechococcus, Synechocystis, Synedra, Synochromonas, Synura, Tabellaria, Tabularia, Teilingia, Temnogametum, Tetmemorus, Tetrachlorella, Tetracyclus, Tetradesmus, Tetraedriella, Tetraedron, Tetraselmis, Tetraspora, Tetrastrum, Thalassiosira, Thamniochaete, Thermosynechococcus, Thorakochloris, Thorea, Tolypella, Tolypothrix, Trachelomonas, Trachydiscus, Trebouxia, Trentepholia, Treubaria, Tribonema, Trichodesmium, Trichodiscus, Trochiscia, Tryblionella, Ulothrix, Uroglena, Uronema, Urosolenia, Urospora, Uva, Vacuolaria, Vaucheria, Volvox, Volvulina, Westella, Woloszynskia, Xanthidium, Xanthophyta, Xenococcus, Zygnema, Zygnemopsis, and Zygonium.

In yet other related embodiments, cells that may be used herein may be one or more cells of a Chloroflexus, Chlownema, Oscillochloris, Heliothrix, Herpetosiphon, Roseiflexus, and Thermomicrobium cell;

a green sulfur bacteria such as: Chlorobium, Clathwchloris, and Prosthecochloris;

a purple sulfur bacteria such as: Allochromatium, Chromatium, Halochromatium, Isochromatium, Marichromatium, Rhodovulum, Thermo chromatium, Thiocapsa, Thiorhodococcus, and Thiocystis;

a purple non-sulfur bacteria such as: Phaeospirillum, Rhodobaca, Rhodobacter, Rhodomicrobium, Rhodopila, Rhodopseudomonas, Rhodothalassium, Rhodospirillum, Rodovibrio, and Roseospira;

an aerobic chemolithotrophic bacteria such as: nitrifying bacteria such as Nitrobacteraceae sp., Nitrobacter sp., Nitrospina sp., Nitrococcus sp., Nitrospira sp., Nitrosomonas sp., Nitrosococcus sp., Nitrosospira sp., Nitrosolobus sp., Nitrosovibrio sp.;

colorless sulfur bacteria such as Thiovulum sp., Thiobacillus sp., Thiomicrospira sp., Thiosphaera sp., Thermothrix sp.;

obligately chemolithotrophic hydrogen bacteria, Hydro genobacter sp., iron and manganese-oxidizing and/or depositing bacteria, Siderococcus sp., and magnetotactic bacteria, Aquaspirillum sp;

an archaeobacteria such as: methanogenic archaeobacteria, Methanobacterium sp., Methanobrevibacter sp., Methanothermus sp., Methanococcus sp., Methanomicrobium sp., Methano spirillum sp., Methanogenium sp., Methanosarcina sp., Methanolobus sp., Methanothrix sp., Methanococcoides sp., Methanoplanus sp.;

extremely thermophilic sulfur-metabolizers such as Thermoproteus sp., Pyrodictium sp., Sulfolobus sp., Acidianus sp., Bacillus subtilis, Saccharomyces cerevisiae, Streptomyces sp., Ralstonia sp., Rhodococcus sp., Corynebacteria sp., Brevibacteria sp., Mycobacteria sp., and oleaginous yeast; and

extremophile such as Pyrolobus fumarii; Synechococcus lividis, mesophiles, psychrophiles, Psychrobacter, Deinococcus radiodurans, piezophiles, bawphiles, hypergravity tolerant organisms, hypogravity tolerant organisms, vacuum tolerant organisms, tardigrades, insects, microbes seeds, dessicant tolerant anhydrobiotic organisms, xerophiles, Artemia salina, nematodes, microbes, fungi, lichens, salt tolerant organisms halophiles, halobacteriacea, Dunaliella salina, pH tolerant organisms, alkaliphiles, Natronobacterium, Bacillus firmus OF4, Spirulina spp., acidophiles, Cyanidium caldarium, Ferroplasma sp., anaerobes, which cannot tolerate O₂, Methanococcus jannaschii, microaerophils, which tolerate some O₂, Clostridium, aerobes, which require O₂, gas tolerant organisms, which tolerate pure CO₂, Cyanidium caldarium, metal tolerant organisms, metalotolerants, or Ferroplasma acidarmanus Ralstonia sp CH34.

### IV. Cell Culturing Methods

In certain aspects, the genetically modified cells may be cultured with a volume of about, at least or at most 0.2 ml, 0.5 ml, 1 ml, 2 ml, 5 ml, 10 ml, 20 ml, 30 ml, 40 ml, 50 ml, 100 ml, 200 ml, 500 ml, 1 liters, 3 liters, 5 liters, 10 liters, 20 liters, 25 liters, 30, liters, 40 liters, 50 liters, or any range derivable therein, such as in a bioreactor. Some embodiments involve cells growing in a space whose volume is larger than a standard petri dish or 96-well plate; consequently, some embodiments exclude the use of such containers.

Culture temperatures may be at ambient temperature (20-25° C.), or may be at 25 to 29° C., or at least, at most, or about 20, 21, 22, 23, 24, 25, 26, 27, 28, 29, 30, 31, 32, 33, 34, 35, 36, 37, 38, 39, 40, 41, 42, 43, 44, 45° C., or any range or value derivable therein. In some embodiments, the cultivation temperatures may reach 35° C. or even as high as 42° C. for limited time periods during the daily cycle. In some embodiments in which the bacteria or algae are heterotrophic or mixotrophic, the culture may be supplemented with energy sources such as glucose or can be grown under darkness.

In some embodiments, cells can be cultured under illumination with bright white and warm fluorescent lights (for example, about 30 to 200 micromol/m²/sec or even to 400 micromol/m²/sec) with, for example, about a 12 hour light:12 hour dark photoperiod or a 14 hour light:10 hour dark photoperiod or a 16 hour light:8 hour dark period, or even a 24 hour light period. In some embodiments, cells can be cultured under natural illumination, with or without supplemental shading in photo bioreactorsL, in covered (closed) culture systems, or in open culture systems such as in raceways and ponds.

In further embodiments, large scale production of cells may be implemented. “Large scale,” as used herein, refers to the use of a cell culture of a volume of at least or about 500 ml, 600 ml, 700 ml, 800 ml, 1 liter, 2 liters, 3 liters, 5 liters, 10 liters, 20 liters, or up to 25 liters, or any range derivable therein, such as in a bioreactor. Cell concentration in a cell culture may be at least or about 10¹, 10², 10³, 10⁴, 10⁵, 10⁶, 10⁷, 10⁸, 10⁹, 10¹⁰ cells/μl, cells/ml, cells/L or any range derivable therein. Cells may also be cultured in low or high densities, such as at least, about or at most 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 g/ml, g/L, mg/L, kg/L, kg/mL, or any derivable value or range thereof.

Cells may be manipulated subsequent to culturing, such as by concentrating them and/or reducing the cell culture volume.

A culture vessel used for culturing the cells can include, but is particularly not limited to: flask, flask for tissue culture, dish, petri dish, dish for tissue culture, multi dish, micro plate, micro-well plate, multi plate, multi-well plate, micro slide, chamber slide, tube, tray, CellSTACK® Chambers, culture bag, and roller bottle, as long as it is capable of culturing the cells therein. The cells may be cultured in a culture vessel with a volume of at least or about 0.2, 0.5, 1, 2, 5, 10, 20, 30, 40, 50 ml, 100 ml, 150 ml, 200 ml, 250 ml, 300 ml, 350 ml, 400 ml, 450 ml, 500 ml, 550 ml, 600 ml, 800 ml, 1000 ml, 1500 ml, or any range derivable therein, depending on the needs of the culture. In a certain embodiment, the culture vessel may be a bioreactor, which may refer to any device or system that supports a biologically active environment. The bioreactor may have a volume of at least or about 2, 4, 5, 6, 8, 10, 15, 20, 25, 50, 75, 100, 150, 200, 500 liters, 1, 2, 4, 6, 8, 10, 15 cubic meters, or any range derivable therein.

Other culturing conditions can be appropriately defined. For example, the culturing temperature can be about 30 to 40° C., for example, at least or about 31, 32, 33, 34, 35, 36, 37, 38, 39° C. but particularly not limited to them, or any range or value derivable therein. The CO₂ concentration can be about 1 to 10%, for example, about 2 to 5%, or any range or value derivable therein. The oxygen tension can be at least or about 1, 5, 8, 10, 20%, or any range or value derivable therein.

Cells such as the photosynthetic cells may grow in an aqueous medium. The aqueous medium can be, for example, tap water, well water, distilled water, reverse osmosis water, filtered water, purified water, sea water, rain water, grey water, river water, lake water, pond water, groundwater, or wastewater. The water can be used unfiltered, unsterilized, or in an unpurified form. Alternatively, the water can be filtered, sterilized, or purified by running through ion exchange columns or charcoal filter, for example.

Nutrients can be added to the aqueous medium, if they are not already present in sufficient quantities to support cyanobacterial or algal growth. Added nutrients can include, but are not limited to, iron, magnesium, calcium, sodium, potassium, phosphorus, sulfur, chloride, and trace metals. The salinity and the pH of the medium can be adjusted, if needed, to suit that of the photosynthetic organism being used.

For example, cells can be cultured in a variety of growth medium. In one embodiment, cells can also be cultured on solidified medium such as by 20 g/L agar, or embedded in solidified medium such as in alginate or other types of matrix including mesh. In some embodiments for liquid culture, the volume is between about 25 mL to 1000 mL. In other embodiments, it can be between about 1 L to about 100 L. In some embodiments, the volume is between about 1 L to about 10 L. In some embodiments, the volume is about 6 L. In some embodiments in outdoor culture, volumes may be about 100 to 600 L, or in larger increments to about 1200 L, 2400 L, and up to about 20,000 L, for example, in bioreactors, including enclosed or covered ponds. In other embodiments, the culture is in 50,000 L raceways or ponds. In yet other embodiments, the culture is in expansive ponds, including in poly culture, of about 1 to 10 acres each.

In certain aspects, an inoculum of genetically modified cells such as cyanobacterial or algal culture is added to the medium. One or more photosynthetic organisms may be present in the inoculum. The medium and cells may be exposed to sun light, to artificial light, or a mixture of natural and artificial light, to allow the phototrophic cells to grow.

This can be accomplished using any type of growth platform including culture flasks, bottles, and tubes, outdoor or indoor raceway ponds, plastic bags or tubes, or a commercial photobioreactor of any design.

The cells may be provided a source of carbon dioxide, such as air, enriched or pure carbon dioxide, flue or combustion gases, or fermentation gases. Some amount of time is allowed to pass, during which cells such as cyanobacterial or algal cells divide and produce biomass. During growth the medium may or may not be circulated using a water wheel, paddle, a pump, or by pumping air or gas through the medium. Finally, once the desired amount of growth has been achieved, the biomass may be harvested.

In certain aspects, static culture may be used. In further aspects, non-static culture could be used, such as suspension culture for large-scale production of cells. The non-static culture can be any culture with cells kept at a controlled moving speed, by using, for example, shaking, rotating, or stirring platforms or culture vessels, particularly large-volume rotating or shaking bioreactors. The agitation may improve circulation of nutrients and cell waste products and also be used to control cell aggregation by providing a more uniform environment. For example, rotary speed may be set to at least or at most about 25, 30, 35, 40, 45, 50, 75, 100 rpm, or any range derivable therein. The incubation period in the non-static culture may be at least or about 4 hours, 8 hours, 16 hours, or 1, 2, 3, 4, 5, 6 days, or 1, 2, 3, 4, 5, 6, 7 weeks, or any range derivable therein.

In some embodiments, any one of the cells culture conditions or methods disclosed herein may be excluded as to the claimed invention.

### V. Cell Disruption Methods

In certain aspects, with the temperature inducible self-destruction strategy described herein, no digestive enzymes need to be added for cell disruption, because the enzymes are produced by the genetically modified cells. For example, three thermostable protease genes were engineered into cyanobacteria Synechocystis PCC 6803 and Synechococcus PCC 7002. The mutant strains autolyzed at elevated temperatures (46-48° C.).

Methods used for the destruction of the cells (for example, cyanobacteria or microalgae) may include increasing the temperature to at least or about 45, 50, 55, 60, 65, 70, 75, 80, 85, 90, 95, 100° C., or any derivable value or range thereof, or any temperature above the temperature of a host cell's culture for producing desired intracellular biological products.

This may allow at least, about or up to 50, 60, 70, 80, 90, or 100% (or any derivable value or range thereof) of cells to lyse in at least, about or up to 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 hours or any derivable value or range thereof.

The temperature increase can be performed using any method known in the art, including, but not limited to, a solar heating system, a wind heating system, a gas heating system, an electrical heating system, a radiation-induced heating system, or a battery-operated heating system, or a heating system that collects heat produced by cells or organisms. For example, this temperature increase can be achieved by solar heating outdoor conditions without any additional cost. The temperature for an enclosed liquid exposed to the sun light in some hot places will increase to 46-49° C. during the day.

In certain aspects, this process may bring down the cost of lysed cells nearly to the cost of wet biomass. One optional advantage in certain aspects may be to reduce the processing cost and to increase the value of microalgae biomass. A list of other optional benefits in further aspects is described below:

1. Genetically Modified Organisms GMO control. As the mutant carrying suicidal genes, they can be grown in defined photo-bioreactors in some hot places. In case of spill, the sun heating in hot places may immediately increase the cell temperatures and cells will undergo self-destruction and die.

2. Overcoming the difficulty of lysing cells in composition analysis. For the bio-portfolio project, this strategy may be used to break up cells for molecule analysis such as lipids, proteins, and carbohydrates.

3. Reducing extraction cost. If there is a need to extract oil or high-value products from rigid cells such as cyanobacterial cells, the conventional solvent extraction method may cost about $1000/ton biomass ($1.75/gal oil). However, this temperature-induced bursting strategy as described herein in certain aspects costs nearly nothing, and will largely reduce the cost of extraction.

4. Peptide supplement for humans. Peptides are hydrolyzed protein fragments, and they are much faster absorbed by human body than other forms of proteins. Peptides are important sport nutrients, as they can reduce the time of recovery compared to proteins. Peptides come with a big price tag. The market price for body-building peptides is over $60,000/ton.

5. Feed or supplement for animals. Pre-digested microalgae are easier for animals to digest and absorb compared to the whole algae cells. In certain aspects, unfavorable components such as DNA (a cause for animal gout) can be reduced by introducing nucleases. At Alibaba, microalgae are sold as feed at >$2,000/ton, and as supplement at >$15,000/ton.

6. Liquid fertilizer. Algae lysate can be used as liquid fertilizers. This is a mature business in China. Liquid algae fertilizers are sold at over $3,000/ton by many Chinese companies.

7. Feedstock for fermentation. Data showed that the cyanobacterial lysates can support E. coli growth. The lysates may replace the sugar feedstock ($400/ton) for growing industrial microorganisms (e.g., the LS9 process).

8. Compatibility. This genetic strategy can be easily applied to other microorganisms such as bacteria, microalgae and yeast using genetic engineering methods known in the art.

In certain aspects, methods and compositions described herein may differentiate from existing techniques that used externally added lytic enzymes to digest algal cells for better product recovery, because the enzymes are all produced and added from outside of the cells. Because of the rigidity of cell envelopes, degrading cells from outside is less efficient than from inside.

One big challenge solved in certain aspects is how to control the activities of lytic enzymes (proteases), because these proteases are able to degrade proteins which are essential for cell viability, and they cannot be largely produced in active form when cells are growing. In certain aspects, this advantage may further distinguish methods and compositions described herein from existing techniques.

Cells for producing desired intracellular biological products may be lysed using methods described herein alone or in combination with any suitable methods known in the state of the art, such as homogenization, solvent extraction (organic phase/aqueous phase), bead mill, freeze/thaw cycles, ultrasonication, additional enzymatic lysis, and the like. In some embodiments, any one of the lysing methods disclosed herein may be excluded as to the claimed invention.

### VI. Biofuel and/or Biomass Production

Methods described herein may include biofuel production related to extracting oil from the cells expressing thermostable proteases. In certain aspects, once the oil has been released from the cells through temperature-induced self-destruction, it can be recovered or separated from the cells, e.g. cellular residue, enzyme, by-products, etc. For example, this can be done by sedimentation or centrifugation, with centrifugation generally being faster. Recovered oil can be collected and directed to a conversion process.

Oil can be converted to biodiesel through a process of direct hydrogenation or transesterification of the oil. For example, Algal oil may be in a similar form as most vegetable oils, which are in the form of triglycerides. This form of oil can be burned directly. However, the properties of the oil in this form may not be ideal for use in a diesel engine. In further aspects, the triglyceride may be converted into biodiesel, which is similar to but superior to petroleum diesel fuel in many respects.

In certain aspects, one process for converting the triglyceride to biodiesel is transesterification, and includes reacting the triglyceride with alcohol or other acyl acceptor to produce free fatty acid esters and glycerol. The free fatty acids are in the form of fatty acid alkyl esters. Transesterification can be done in several ways, including biologically and/or chemically. The biological process may use an enzyme known as a lipase to catalyze the transesterification, while the chemical process may use, but is not limited to, a synthetic catalyst that may be either an acid or a base. With the chemical process, additional steps are needed to separate the catalyst and clean the fatty acids. In addition, if ethanol is used as the acyl acceptor, it may be dried to prevent production of soap via saponification in the process, and the glycerol may be purified. Either or both of the biological and chemically-catalyzed approaches can be useful in connection with the processes described herein.

Further, the biomass may contain valuable components such as proteins and carbohydrates. Proteins from the biomass can serve as a source for growth hormone peptides, disease resistance compounds, vaccines, and therapeutic proteins. In addition, proteins from the biomass can serve as promising sources for various industrial applications.

In further aspects, biomass production can follow similar separation processes. For example, the biomass left after the oil is separated may be fed into the depolymerization to recover any residual energy by conversion to sugars, and the remaining cell residues can be either burned for processing heat or sold as an animal food supplement or fish food.

Lipids extracted from harvested biomass may be further processed. In one embodiment, esterification is performed to allow analysis by gas chromatography of fatty acid methyl esters, as is well-known to those skilled in the art. In another embodiment, lipids are hydrolyzed (saponified). In another embodiment, saponification or esterification can be applied either concurrent with, or subsequent to, the actual lipid extraction from the biomass.

Proteins extracted from harvested biomass can also be further processed. For bioactive proteins, the protein conformation may be preserved during processing. Nutritional quality of proteins used for feed is determined based on the content, proportion, and availability of amino acids. Analysis of free amino acids can be processed by high performance liquid chromatography, as is known in the art.

In some embodiments, any one of the biofuel and/or biomass production methods disclosed herein may be excluded as to the claimed invention.

### VII. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Design and Synthesis of Thermostable Protease Genes

The highly expressed genes in the Synechocystis PCC6830 and Synechococcus PCC7002 were analyzed, and a codon usage bias table for host cyanobacteria was built (Table 3).

TABLE 3
Synechococcus sp. PCC 7002 and Synechocystis PCC6803 highly expressed gene codon bias
fields: [triplet] [amino acid] [fraction] [frequency: per thousand]

| | | | | | | | | | | | | | | | |
|---|---|---|---|---|---|---|---|---|---|---|---|---|---|---|---|
| UUU | F | 0.67 | 30.2 | UCU | S | 0.18 | 10.1 | UAU | Y | 0.56 | 17.6 | UGU | C | 0.60 | 7.2 |
| UUC | F | 0.33 | 15.0 | UCC | S | 0.20 | 11.2 | UAC | Y | 0.44 | 13.8 | UGC | C | 0.40 | 4.7 |
| UUA | L | 0.16 | 17.5 | UCA | S | 0.07 | 4.0 | UAA | * | 0.56 | 1.9 | UGA | * | 0.15 | 0.5 |
| UUG | L | 0.19 | 21.2 | UCG | S | 0.13 | 7.0 | UAG | * | 0.28 | 0.9 | UGG | W | 1.00 | 14.6 |
| CUU | L | 0.10 | 11.5 | CCU | P | 0.16 | 7.7 | CAU | H | 0.47 | 9.4 | CGU | R | 0.21 | 10.4 |
| CUC | L | 0.27 | 30.2 | CCC | P | 0.47 | 23.3 | CAC | H | 0.53 | 10.6 | CGC | R | 0.35 | 17.4 |
| CUA | L | 0.09 | 9.6 | CCA | P | 0.15 | 7.3 | CAA | Q | 0.59 | 29.5 | CGA | R | 0.10 | 4.8 |
| CUG | L | 0.19 | 21.6 | CCG | P | 0.22 | 11.0 | CAG | Q | 0.41 | 20.5 | CGG | R | 0.28 | 13.8 |
| AUU | I | 0.56 | 35.9 | ACU | T | 0.15 | 8.5 | AAU | N | 0.61 | 21.1 | AGU | S | 0.21 | 11.8 |
| AUC | I | 0.42 | 26.9 | ACC | T | 0.48 | 27.7 | AAC | N | 0.39 | 13.6 | AGC | S | 0.20 | 11.1 |
| AUA | I | 0.02 | 1.2 | ACA | T | 0.15 | 8.5 | AAA | K | 0.66 | 27.4 | AGA | R | 0.05 | 2.3 |
| AUG | M | 1.00 | 22.9 | ACG | T | 0.22 | 12.4 | AAG | K | 0.34 | 14.0 | AGG | R | 0.03 | 1.4 |
| GUU | V | 0.24 | 16.5 | GCU | A | 0.18 | 15.9 | GAU | D | 0.69 | 31.9 | GGU | G | 0.30 | 22.0 |
| GUC | V | 0.27 | 18.9 | GCC | A | 0.42 | 37.3 | GAC | D | 0.31 | 14.3 | GGC | G | 0.34 | 25.1 |
| GUA | V | 0.11 | 7.9 | GCA | A | 0.14 | 12.7 | GAA | E | 0.76 | 44.1 | GGA | G | 0.11 | 8.4 |
| GUG | V | 0.37 | 25.9 | GCG | A | 0.26 | 23.5 | GAG | E | 0.24 | 13.8 | GGG | G | 0.25 | 18.8 |

By using the codon bias table avoiding low frequency triplets (below 0.10 at cutoff), and avoiding 5′-RNA stem loop structures, the protease genes and other DNA parts were designed and synthesized. The protease genes are designed in a unified form with 5′ flanked by Ptrc promoter and RBS (Sequence: agTACGTATTGACAATTAATCATCCGGCTCGTATAATATTAAAGAGGAGAAAcatATG; SEQ ID NO.: 1), and 3′ flanked by a multi-cloning site (Sequence: TAATGGATCCAAGATCTCGAATTCTAAGCTTCGATATCGACTAGTag; SEQ ID NO.: 2). Some of the designs are shown in SEQ ID NOs.: 13-16. Also, a selection marker cassette SacB KmR was synthesized for screening positive colonies (SEQ ID NO.: 17).

### Example 2—Construction of DNA Fragments and Transformation for Cyanobacteria

Linear DNA fragments were constructed by splicing overlapping DNA sequences via Gibson Assembly and PCR (See, for example, Gibson, et al., Nature Methods, 343-345, 2009). Taking the transformation of Synechococcus 7002 as an example, four overlapping pieces were amplified from the genomic DNA or synthesized template by PCR: Piece 1 [left flanking region], Piece 2 [Ptrc+Gene of Interest], Piece 3 [multi-cloning site+KmR], and Piece 4 [right flanking region]. The primers used for PCR are listed in Table 4.

The transformation of Synechocystis PCC6803 was similar to the protocol, the main difference was that Synechocystis PCC6803 was a freshwater cyanobacterium, and culture medium for Synechocystis PCC6803 was fresh water BG-11 (ATCC® Medium 616).

TABLE 4
Primers used for Synechococcus 7002 transformation

| | | SEQ | |
|---|---|---|---|
| | | ID | |
| Name | Sequence (5′ to 3′) | NO.: | Note |
| 0053S71F1S | AGC GAT GCG AAT ATT CAT GCC | 3 | Amplifying [left |
| | GAC TAA C | | flanking region] |
| 0065S71F1A-to- | GAT GAT TAA TTG TCA AAT CGA | 4 | Amplifying [left |
| Ptrc | TGC CGA AAT CAT GGC TAC AAT C | | flanking region] |
| 0066PtrcS-to- | GAT TGT AGC CAT GAT TTC GGC | 5 | Amplifying [P_{trc} + Gene |
| S71F1 | ATC GAT TTG ACA ATT AAT CAT C | | of Interest] |
| 0026MCS-A-to- | AGA TTT TGA GAC ACA ACG TGG | 6 | Amplifying [P_{trc} + Gene |
| KmR | CTT TAC TAG TCG ATA TCG AAG CTT | | of Interest] |
| | AGA A | | |
| 0027KmR-S-to- | TTC TAA GCT TCG ATA TCG ACT AGT | 7 | Amplifying [multi- |
| MCS | AAA GCC ACG TTG TGT CTC AAA | | cloning site + KmR] |
| | ATC T | | |
| 0067KmRA-to- | AAT TTG ATC GAT GTA GGG ACT | 8 | Amplifying [multi- |
| S71F2 | GCT GAT TAG AAA AAC TCA TCG AG | | cloning site + KmR] |
| 0068S71F2S-to- | CTC GAT GAG TTT TTC TAA TCA GCA | 9 | Amplifying [right |
| KmR | GTC CCT ACA TCG ATC AAA TT | | flanking region] |
| 0056S71F2A | GGA GTT GGA TAT TTT CCA TTT GTT | 10 | Amplifying [right |
| | GAG CGA CCT | | flanking region] |
| 0057S71SegS | GTC TTC GTC AGG AAA TGT TGA | 11 | Sequencing primer for |
| | TGG CGA TC | | insertions |
| 0058S71SegA | GGG CTT CGA GGT TCG GCA CAA | 12 | Sequencing primer for |
| | TTA A | | insertions |

Brief protocol for construction DNA fragments for transformation:

Step 1:

Amplify four pieces of DNA parts. Piece 1 and Piece 4 were amplified from 7002 genomic DNA, Piece 2 was amplified from synthesized protease genes (SEQ ID NOs.: 13-16), and Piece 3 was amplified from the SacB KmR cassette (SEQ ID NO.: 17).

Step 2:

The purified overlapping PCR products was collected, and spliced them by Gibson Assembly (Gibson Assembly® Master Mix available from New England Biolabs Inc. Catalog # E2611L). About 20 ng of each DNA piece was mixed with 2× Gibson Assembly® Master Mix, and incubated at 50° C. for one hour.

Step 3:

The whole DNA construct was amplified by using end primers (0053S71F1S and 0056S71F2A). The PCR products were purified by electrophoresis.

After DNA construction, Synechococcus transformation was performed by a protocol below:

Step 1:

30 μl Synechococcus culture (OD730 nm between 0.5 and 1) was mixed with 100-300 ng DNA fragments (Purified PCR products), incubated at 30° C. for 4 hours, and then the mixture was inoculated into 2 ml MN Marine medium (ATCC Medium 957 based on Red Sea water).

Step 2:

The culture was grown on a 150 rpm shake with 50 μE/m²/s light intensity at 30° C. for 2 days for segregation.

Step 3:

100 μl and 500 μl cultures were spread onto MN medium agar plates supplemented with 50 mg/l kanamycin. The plates were incubated with 50 μE/m²/s light intensity at 30° C. The colonies usually appeared appear in one week.

Step 4:

The individual colonies were streaked onto a new MN plate with kanamycin and grown for one week. A visible amount of cells were tipped into 2-3 μl water. The cells were frozen at −80° C. and thawed at 50° C. warm water, and then repeated for 3 cycles. The freeze-thaw cells were used as a template and primers 0057S71SegS and 0058S71SegA were used for PCR amplification. The correct insertion of gene of interest was confirmed by sequencing.

### Example 3—Protease-Incorporated Synechocystis Self-Destruction at Elevated Temperatures

By the methods described above, two protease genes tap from Thermoactinomyces sp. E79 (SEQ ID NO.: 15) and gsp from Geobacillus stearothermophilus F1 (SEQ ID NO.: 16) were inserted into Synechocystis PCC6803 genome at the fadD site, which resulted in two mutant strains SAB303 (ΔfadD::P_{trc} tap Km^{R}) and SAB304 (ΔfadD::P_{trc} gsp Km^{R}).

50 μl of cultures (OD_{730 nm}˜0.7) of the mutant cells and the wild type cells were incubated on a temperature gradient of 56° C., 52° C., 48° C., 44.3° C., 40.5° C., 37° C. and 25° C. The cell viability was checked by SYTOX Green staining (LifeTechnologies catalogue number S7020) and fluorescence microscopy (excitation at 488 nm, and emission at 509 nm). The cells fluorescing green were recorded as dead cells.

As shown in Table 5, the mutant strains SAB303 and SAB304 showed higher death rate than the wild type, when incubated at elevated temperatures for one day and two days.

TABLE 5
Death percentage of Synechocystis PCC6803 and mutants with
protease genes at elevated temperatures

| | Temperature | | | |
|---|---|---|---|---|
| Time | (° C.) | Wild type | SAB303 (tap) | SAB304 (gsp) |
| Day 0 | 30 | <1% | <1% | <1% |
| Day 1 | 25 | <1% | <1% | <1% |
| | 37 | 4% | 7% | 9% |
| | 40.5 | 1.4% | 9% | 9% |
| | 44.3 | 7.6% | 11% | 4% |
| | 48 | 9% | 52% | 14% |
| | 52 | 13.4% | 100% | 37.5% |
| | 56 | 13% | 100% | 89% |
| Day 2 | 25 | <1% | <1% | <1% |
| | 37 | 3% | 3% | 2% |
| | 40.5 | 4% | 42% | 2% |
| | 44.3 | 20% | 94% | 11.9% |
| | 48 | 31% | 100% | 85.7% |
| | 52 | 80% | 100% | 92% |
| | 56 | 89% | 100% | 100% |

### Example 4—Synechocystis Cell Lysate Supporting the Growth of E. coli

Thermostable proteases can degrade the protein component of the cells into small peptides or amino acids when activated at higher temperature. In the meanwhile, the carbohydrates (e.g., glycogen) in the cells will be released. To test the nutritional value of cell lysates, the lysates were inoculated with E. coli cells as culture broth.

SAB303, SAB304 and Synechocystis 6803 cultures were concentrated to about 0.2 g/ml. One portion was treated at 48° C. for 2 days; another portion without treatment was left at room temperature. 10 μl and 100 μl lysates were added to 2 ml E. coli suspension (OD₆₀₀ nm=0.008, 5.2±0.2×10⁵/ml)) in M9 medium (without carbon source). The mixtures were incubated in a shaking incubator at 37° C. for 12 hours. The E. coli cell titers were measured by plating dilutes onto LB plates and counting colony forming unit.

As shown in Table 6, the mutant cell lysates treated at elevated temperature supported the growth of E. coli cells more than the untreated cells. It was also observed that the E. coli cells grew better (25-40%) with the heat-treated mutant cells than with the heat-treated wild type cells. This suggested that the temperature induced self-digested cells could provide nutrient for the growth of heterotrophic bacteria such as E. coli.

TABLE 6
E. Coli growth supported by treated synechocystis biomass

| | Added with Untreated | | Added with Heat-treated | |
|---|---|---|---|---|
| | Synechocystis biomass | | Synechocystis biomass | |
| | 10 μl | 100 μl | 10 μl | 100 μl |
| | concentrates | concentrates | lysates | lysates |
| Wild type | 6.1 ± 1.1 | 9.4 ± 0.2 | 24 ± 0.7 | 107.4 ± 15.9 |
| | (10⁵/ml) | (10⁵/ml) | (10⁵/ml) | (10⁵/ml) |
| SAB303 (tap) | 10.3 ± 3.2 | 5.3 ± 1.1 | 22.1 ± 0.7 | 120.0 ± 10.1 |
| | (10⁵/ml) | (10⁵/ml) | (10⁵/ml) | (10⁵/ml) |
| SAB304 (gsp) | 6.1 ± 1.1 | 11.6 ± 3.5 | 30.4 ± 0.3 | 148.4 ± 2.8 |
| | (10⁵/ml) | (10⁵/ml) | (10⁵/ml) | (10⁵/ml) |

### Example 5—Protease-Incorporated Synechococcus Self-Destruction at Elevated Temperatures

Protease gene ttp from Thermoanaerobacter tengcongensis was inserted into marine cyanobacterium Synechococcus PCC7002, by the method mentioned above. The mutant strain SAB406 (ΔfadD::P_{trc} ttp Km^{R}) was grown at 30° C. 50 μl of cultures (OD_{730 nm}˜0.7) of the mutant cells and the wild type cells were incubated on a temperature gradient of 48° C., 46.4° C., 44.1° C., 41.2° C., 37° C., 33.3° C., 31.1° C. and 30° C. The treatment time period is 24 hours. The cell viability was checked by SYTOX Green staining (LifeTechnologies catalogue number S7020) and fluorescence microscopy (excitation at 488 nm, and emission at 509 nm). The cells fluorescencing green were recorded as dead cells.

As shown in Table 7, protease ttp from Thermoanaerobacter tengcongensis was activated at elevated temperatures and caused self-destruction of Synechococcus 7002 cells. In 24 hours period of time, 100% of mutant cells became dead at 46.4° C., while the death rate of wild type cells is less than 50%. This confirmed the concept that thermostable protease ttp worked at 46.4° C. and killed 100% cells in one day.

TABLE 7
Death percentage of Synechococcus PCC7002 and
SAB406 with protease ttp at elevated temperatures

| Temperature | | |
|---|---|---|
| (° C.) | Wild type | SAB406 (ttp) |
| 48 | 52% | 100% |
| 46.4 | 46% | 100% |
| 44.1 | 12% | 32% |
| 41.2 | 3% | 11% |
| 37 | 2% | 9% |
| 33.3 | 2% | 5% |
| 31.1 | 1% | 2% |
| 30 | 1% | 1% |

### Example 6—Synechococcus Cell Lysate Supporting the Growth of E. coli

The nutritional benefit of Synechococcus cell lysate was tested with the growth of E. coli. Briefly, wild type 7002 and mutant SAB406 were grown to cell density of about 3 g/l of biomass, and then concentrated to about 50 g/l by centrifugation. One portion (1 ml) was treated at 48° C. for 24 hours; another portion (1 ml) without treatment was left at room temperature.

E. coli cells suspension were added to the untreated cell concentrates and heat-treated lysate to give initial cell concentrations of 10⁵ cells/ml and 10⁶ cells/ml. The mixtures were incubated in a shaking incubator (250 rpm) for 20 hours at 37° C. The E. coli cell titers were measured by plating dilutes onto LB plates and counting colony forming unit.

As shown in Table 8, comparing to untreated cells, the heat-treated SAB406 mutant cells supported the growth of E. coli to a greater extent. However, the nutritional advantage of wild type 7002 cells was not significant, suggesting that inducible self-destruction was useful for releasing nutrition for supporting the growth of heterotrophic bacteria.

TABLE 8
E. coli growth supported by treated Synechococcus biomass

| | E. coli cell titer | | | |
|---|---|---|---|---|
| | Untreated cell, kept at room | | Heat-treated cells at 46° C. for 24 | |
| | temperature | | hours | |
| Initial Inoculum | 10⁵ cells/ml | 10⁶ cells/ml | 10⁵ cells/ml | 10⁶ cells/ml |
| Growth | 7.5 × 10⁵ cells/ml | 4.5 × 10⁶ cells/ml | <10⁵ cells/ml | 3 × 10⁷ cells/ml |
| with7002 wild | | | | |
| type for 20 hr | | | | |
| Growth with | <10⁵ cells/ml | 3 × 10⁶ cells/ml | 7 × 10⁷ cells/ml | 8 × 10⁷ cells/ml |
| SAB406 for 20 hr | | | | |

### Example 7—Protease-Incorporated Synechococcus Strains Grow Normally Under Outdoor Growth Conditions

Protease gene ttp from Thermoanaerobacter tengcongensis was inserted into marine cyanobacterium Synechococcus PCC7002, by the method mentioned above. The mutant strain SAB407 (ΔfadD::P_{trc} tap Km^{R}) and the previously described SAB406 (ΔfadD::P_{trc} ttp Km^{R}) strain were grown in 3-liter bubble columns aerated with 1% CO2 enriched air to test the growth of the Synechococcus cultures under outdoor conditions.

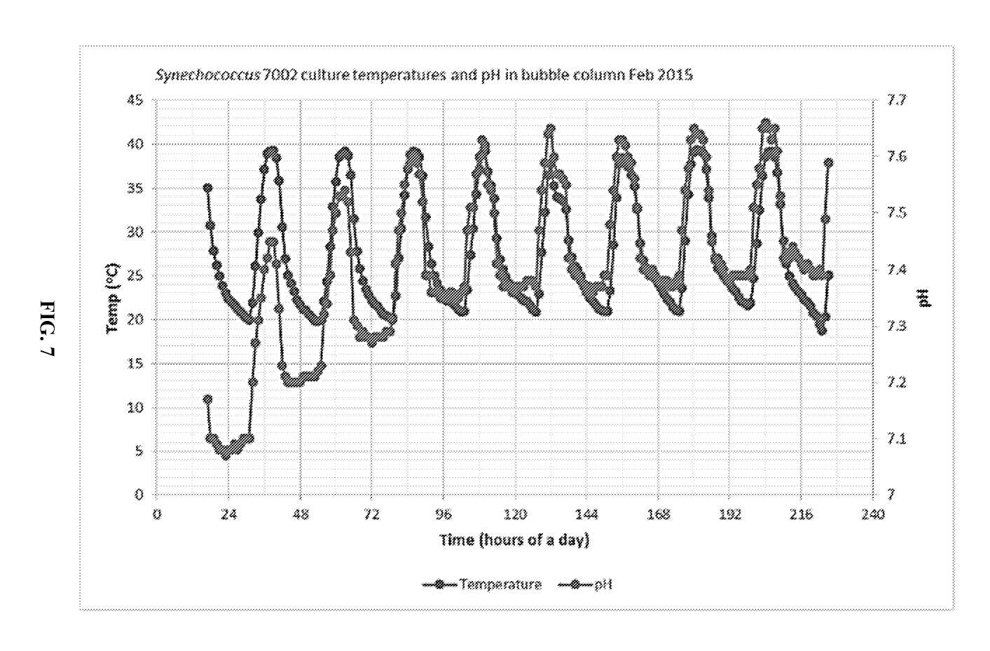

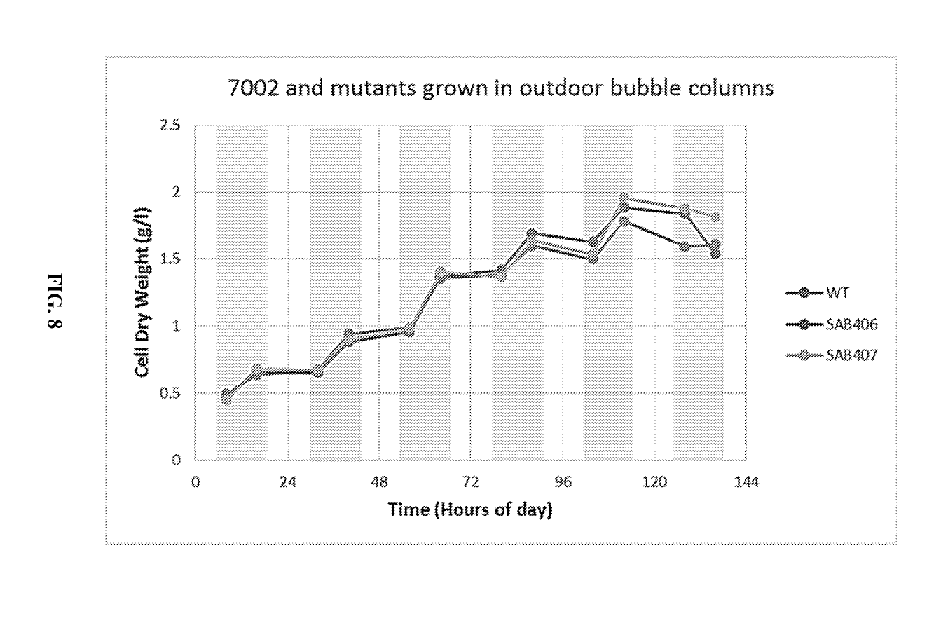

The experiments were performed in February in the Kingdom of Saudi Arabia. Without applying cooling, the culture temperatures varied between 20-40° C. during the day-night shift (FIG. 7). As shown in FIG. 8, the growth curves of the mutant cells were similar to that of wild type Synechococcus PCC7002.

Cell viability was checked by SYTOX Green staining (LifeTechnologies catalogue number S7020) and fluorescence microscopy (excitation at 488 nm, and emission at 509 nm) (see, for example, Liu, et al., Proceedings of the National Academy of Sciences. 108(17):6905-6908, 2011). The cells fluorescing green were recorded as dead cells. The percentage of lysed cells in the cultures was below 5% for all cultures growing in the bubble columns. This observation suggested no significant self-destruction happened in the cultures grown in the bubble columns.

### Example 8—Protease-Incorporated SAB406 (Ttp) and SAB407 (Tap) Self-Destruction at Elevated Temperatures

50 μl of cultures (OD_{730 nm}˜0.7) of the SAB407 (ΔfadD::P_{trc} tap Km^{R}), SAB406 (ΔfadD::P_{trc} ttp Km^{R}), and wild type cells were incubated on a temperature gradient of 50° C., 47° C., 45° C., 41° C., 37° C., 35° C. and 32° C. for 24 hours. The cell viability was checked by SYTOX Green staining and fluorescence microscopy. As shown in Table 9, the mutant strains SAB406 and SAB407 showed higher death rate than the wild type, when incubated at elevated temperatures for one day. This result suggested that the thermostable proteases became active and destructed the cells at elevated temperatures.

TABLE 9
Temperature-induced lysis percentage of Syenchococcus
cells at elevated temperatures

| Temperature | | SAB406 | |
|---|---|---|---|
| (° C.) | Wild type | (ttp) | SAB407 (tap) |
| 50 | 50% | 100% | 100% |
| 47 | 45% | 100% | 90% |
| 45 | 12% | 35% | 25% |
| 41 | 3% | 10% | 8% |
| 37 | 2% | 9% | 4% |
| 34 | 2% | 5% | 3% |
| 32 | 1% | 2% | 1% |

### Example 9—SAB406 (Ttp) and SAB407 (Tap) Cell Lysates Supporting the Growth of E. coli

Thermostable proteases should degrade protein components of a cell into small peptides or amino acids when activated at higher temperature. At the same time, the carbohydrates (e.g., glycogen) in the cells will be released. To test the nutritional value of cell lysates, the lysates were inoculated with E. coli cells as culture broth.

SAB406, SAB407 and Synechcoccus 7002 cultures were concentrated to about 0.2 g/ml. One portion was treated at 48° C. for 2 days; another portion without treatment was left at room temperature. 10 μl and 100 μl lysates were added to 2 ml E. coli suspension (OD600 nm=0.008, 5.2±0.2×10⁵/ml)) in M9 medium (without carbon source). The mixtures were incubated in a shaking incubator at 37° C. for 12 hours. The E. coli cell titers were measured by plating dilutes onto LB plates and counting colony forming unit.

As shown in Table 10, the mutant cell lysates treated at elevated temperature supported the growth of E. coli cells more than the untreated cells. It was also observed that the E. coli cells grew better with the heat-treated mutant cells than with the heat-treated wild type cells. This suggests that the temperature induced self-digested cells can provide nutrient for the growth of heterotrophic bacteria such as E. coli.

TABLE 10
E. coli growth supported by treated Synechococcus biomass

| | c.f.u. (10⁵/ml) | | | |
|---|---|---|---|---|
| | Added with Untreated | | Added with Heat-treated | |
| | Synechococcus biomass | | Synechococcus biomass | |
| | 10 μl lysate | 100 μl lysate | 10 μl lysate | 100 μl lysate |
| Control (lysate | 0 | 0 | 0 | 0 |
| without E. coli) | | | | |
| 7002 (wild | 5.1 ± 1.4 | 9.4 ± 0.2 | 15 ± 2.7 | 59.4 ± 13.9 |
| type) | | | | |
| SAB406 (ttp) | 10.3 ± 4.2 | 5.3 ± 1.1 | 25.6 ± 3.7 | 145.0 ± 16.1 |
| SAB407 (tap) | 8.2 ± 1.1 | 16.6 ± 2.4 | 35.4 ± 4.3 | 138.4 ± 9.8 |

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. An engineered photosynthetic bacteria cell comprising a heterologous nucleic acid segment encoding a thermostable protease.

2. The engineered photosynthetic bacteria cell of claim 1, wherein the heterologous nucleic acid segment is integrated into the bacterial genome.

3. The engineered photosynthetic bacteria cell of claim 1, wherein the thermostable protease is from a bacteria.

4. The engineered photosynthetic bacteria cell of claim 3, wherein the thermostable protease is from a thermophilic bacteria.

5. The engineered photosynthetic bacteria cell of claim 1, wherein the thermostable protease is App (Aquifex pyrophilus), Ttp (Thermoanaerobacter tengcongensis), Tap (Thermoactinomyces sp. E79), Tfp (Thermomonospora fusca), gsp (Geobacillus stearothermaphilus F1), PFUL (Pyrococcus furiosus DSM3638), npr (Bacillus caldolytics), stp (Sulfolobus acidocaldarius), nprM (Bacillus stearothermaphilus), TtHB (Thermus thermophilus HB8), scpA (Alicyclobacillus sendaiensis), pro (Chaetomium thermophilum), bstp (Bacillus stearothermophilus), Afp (Aspergillus fumigatus WY-2), nprB (Bacillus subtilis WB30), or tau (Thermoascus aurantiacus).

6. The engineered photosynthetic bacteria cell of claim 1, wherein the heterologous nucleic acid segment comprises a selectable or screenable marker.

7. The engineered photosynthetic bacteria cell of claim 1, wherein the thermostable protease is under the transcriptional control of a promoter.

8. The engineered photosynthetic bacteria cell of claim 1, wherein the thermostable protease is not from a bacteriophage.

9. The engineered photosynthetic bacteria cell of claim 1, wherein the photosynthetic bacteria is aerobic during photosynthesis.

10. The engineered photosynthetic bacteria cell of claim of 1, wherein the photosynthetic bacteria cell is cyanobacteria.

11. The engineered photosynthetic bacteria cell of claim 1, wherein the cell is an engineered Synechocystis PCC6803 cell and wherein the heterologous nucleic acid segment encodes Tap (Thermoactinomyces sp. E79).

12. The engineered photosynthetic bacteria cell of claim 1, wherein the cell is an engineered Synechocystis PCC6803 cell and wherein the heterologous nucleic acid segment encodes gsp (Geobacillus stearothermaphilus F1).

13. The engineered photosynthetic bacteria cell of claim 1, wherein the cell is an engineered Synechococcus PCC7002 cell and wherein the heterologous nucleic acid segment encodes Tap (Thermoactinomyces sp. E79).

14. The engineered photosynthetic bacteria cell of claim 1, wherein the cell is an engineered Synechococcus PCC7002 cell and wherein the heterologous nucleic acid segment encodes Ttp (Thermoanaerobacter tengcongensis).

15. A cell lysate comprising lysed photosynthetic bacteria cells of claim 1.

16. A method for inducing lysis of photosynthetic bacteria cells comprising exposing the bacteria cells to an elevated temperature between about 45° C. and about 100° C., wherein the bacteria cells comprise photosynthetic bacteria cells of claim 1.

17. The method of claim 16, further comprising collecting cell lysate from the photosynthetic bacterial cells.

18. The method of claim 16, wherein exposed photosynthetic bacterial cells are lysed and resulting cell lysate is exposed to bacteria that has not been exposed to elevated temperatures.

19. The method of claim 16, wherein cell lysis is not induced significantly by exposure to carbon dioxide or metal.

20-24. (canceled)

25. The method of claim 16, wherein the photosynthetic bacteria cell is cyanobacteria.
